(19) United States
(12) Patent Application Publication
(10) Pub. No.: US 2025/0099728 A1
(43) Pub. Date: Mar. 27, 2025

(54) CARDIOVASCULAR SHUNT DEVICES WITH FLOW CONDITIONERS TO MINIMIZE DISRUPTION TO AND ENHANCE CARDIOVASCULAR HEMODYNAMICS

(71) Applicant: EDWARDS LIFESCIENCES CORPORATION, Irvine, CA (US)

(72) Inventors: Mazen Saadi Albaghdadi, Irvine, CA (US); Brandon Sina Dehdashtian, Irvine, CA (US); John Charles Laschinger, Laguna Beach, CA (US)

(21) Appl. No.: 18/969,612

(22) Filed: Dec. 5, 2024

Related U.S. Application Data
(63) Continuation of application No. PCT/US2023/024704, filed on Jun. 7, 2023.
(60) Provisional application No. 63/349,828, filed on Jun. 7, 2022.

(51) Int. Cl. A61M 27/00

(52) U.S. Cl. CPC A61M 27/002; A61M 2205/0216; A61M 2205/025; A61M 2205/0266; A61M 2205/04; A61M 2205/3331; A61M 2206/20; A61M 2210/125

(57) ABSTRACT

A shunt device includes a shunt body formed of a plurality of struts and a flow conditioner connected to the plurality of struts of the shunt body. The shunt body includes a central flow tube, a flow path extending through the central flow tube, and a plurality of arms extending outwardly from the central flow tube for securing the shunt device to a tissue wall. The flow conditioner is positioned to modify a hemodynamic characteristic of a flow of blood through or out of the central flow tube. A control system may be provided for adjusting the position of a flow conditioner after implantation in the body, thereby providing a mechanism for further enhancing cardiovascular hemodynamics.

### CROSS-REFERENCE TO RELATED APPLICATIONS

This application is a continuation of International Application No. PCT/US2023/024704, filed Jun. 7, 2023, which claims the benefit of U.S. Provisional Application No. 63/349,828, filed Jun. 7, 2022, the disclosures of which are hereby incorporated by reference in their entireties.

### BACKGROUND

The present disclosure relates to cardiovascular shunt devices, and more specifically to cardiovascular shunt devices for minimizing disruption to and enhancing cardiovascular hemodynamics.

Shunt devices can be positioned in the heart to shunt blood between the left atrium and the right atrium to reduce pressure in the left atrium. The left atrium can experience elevated pressure due to abnormal heart conditions caused by age and/or disease. For example, shunt devices can be used to treat patients with heart failure (also known as congestive heart failure). Shunt device can be positioned in the septal wall between the left atrium and the right atrium to shunt blood from the left atrium into the right atrium, thus reducing the pressure in the left atrium.

### SUMMARY

In one example, a shunt device includes a shunt body formed of a plurality of struts and a flow conditioner connected to the plurality of struts of the shunt body. The shunt body includes a central flow tube, a flow path extending through the central flow tube, and a plurality of arms extending outward from the central flow tube and configured to secure the shunt device to a tissue wall. The flow conditioner is positioned to modify a hemodynamic characteristic of a flow of blood through or out of the central flow tube.

### BRIEF DESCRIPTION OF THE DRAWINGS

FIG. 1 is a schematic diagram of a heart and vasculature.

FIG. 2 is a cross-sectional schematic view of the heart.

FIG. 3A is a first schematic diagram illustrating modeled hemodynamic flow patterns in a heart.

FIG. 3B is a second schematic diagram illustrating modeled hemodynamic flow patterns in a heart.

FIG. 4A is a first schematic diagram illustrating modeled hemodynamic flow patterns in a heart with a septal shunt device.

FIG. 4B is a second schematic diagram illustrating modeled hemodynamic flow patterns in a heart with a septal shunt device.

FIG. 5A is a first schematic diagram illustrating modeled hemodynamic flow patterns in a heart with a left atrium to coronary sinus shunt device.

FIG. 5B is a second schematic diagram illustrating modeled hemodynamic flow patterns in a heart with a left atrium to coronary sinus shunt device.

FIG. 6A is a perspective view of a shunt device.

FIG. 6B is a side view of the shunt device.

FIG. 6C is a bottom view of the shunt device.

FIG. 7 is a perspective view of the shunt device in a collapsed configuration.

FIG. 8 is a side view of the shunt device with a sensor and anchored to a tissue wall.

FIG. 9 is a bottom view of a first example of a shunt device including fin-type flow conditioners.

FIG. 10 is a side view of the first example of the shunt device anchored to a tissue wall and including the fin-type flow conditioners.

FIG. 11A is an enlarged partial perspective view illustrating a fin-type flow conditioner interacting with low blood flow through the first example of the shunt device.

FIG. 11B is an enlarged partial perspective view illustrating the fin-type flow conditioner interacting with high blood flow through the first example of the shunt device.

FIG. 12A is a schematic diagram illustrating connection of control components to an actively controlled flow conditioner.

FIG. 12B is a schematic diagram illustrating electromechanical actuation of the actively controlled flow conditioner.

FIG. 13 is a sectional view of a heart illustrating an example positioning of the control components for the actively controlled flow conditioner of FIGS. 12A-12B.

FIG. 14 is a schematic diagram illustrating an example control system for the actively controlled flow conditioner of FIGS. 12A-12B.

FIGS. 15A-15E are enlarged partial perspective views of a frame of the first example of the shunt device illustrating several variations of fin-type flow conditioners.

FIG. 16 is a bottom view of a second example of a shunt device including plate-type flow conditioners.

FIG. 17 is a side view of the second example of the shunt device anchored to a tissue wall and including the plate-type flow conditioners.

FIGS. 18A-18E are bottom views of the second example of the shunt device illustrating several variations of plate-type flow conditioners.

FIGS. 19A-19C are side views of a third example of a shunt device anchored to a tissue wall and illustrating several variations of deflector-type flow conditioners for directing fluid flow in one direction.

FIGS. 20A-20C are side views of a fourth example of a shunt device anchored to a tissue wall and illustrating several variations of deflector-type flow conditioners for directing fluid flow in multiple directions.

FIG. 21 is a flowchart showing a method for selecting a shunt device including a flow conditioner for implantation in the heart.

### DETAILED DESCRIPTION

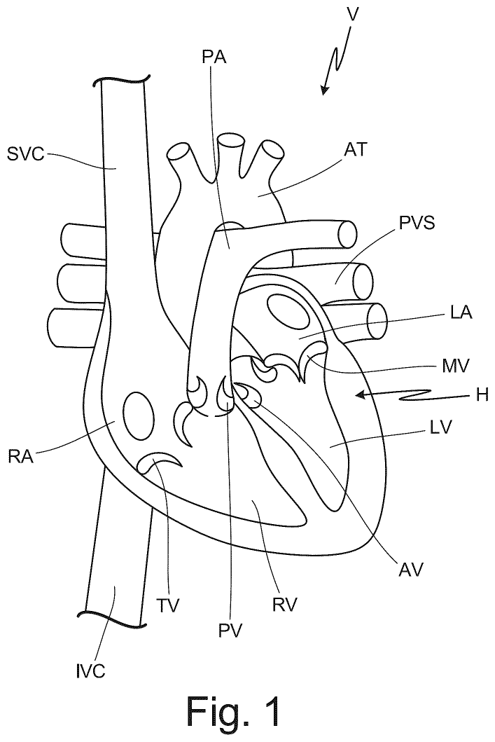

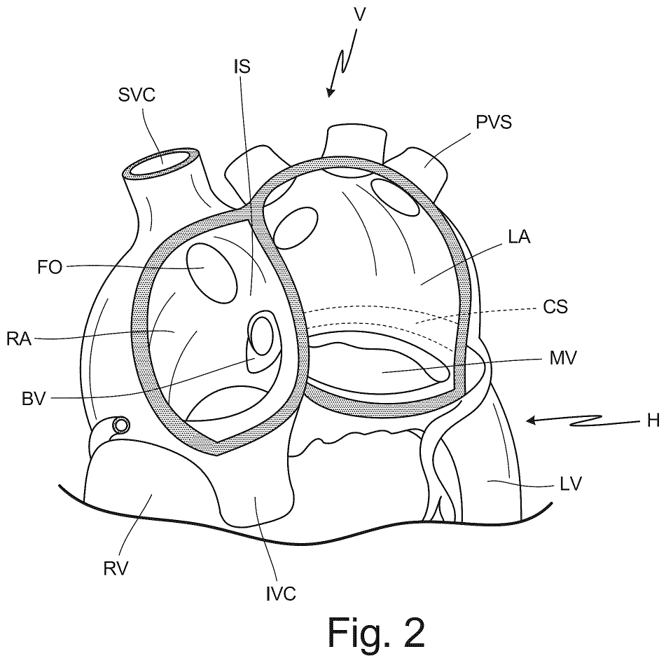

FIG. 1 is a schematic diagram of heart H and vasculature V. FIG. 2 is a cross-sectional schematic view of heart H. FIGS. 1-2 will be discussed together. FIGS. 1-2 show heart H, vasculature V, right atrium RA, right ventricle RV, left atrium LA, left ventricle LV, superior vena cava SVC, inferior vena cava IVC, tricuspid valve TV (shown in FIG. 1), pulmonary valve PV (shown in FIG. 1), pulmonary artery PA (shown in FIG. 1), pulmonary veins PVS, mitral valve MV, aortic valve AV (shown in FIG. 1), aorta AT (shown in FIG. 1), coronary sinus CS (shown in FIG. 2), thebesian valve BV (shown in FIG. 2), inter-atrial septum IS (shown in FIG. 2), and fossa ovalis FO (shown in FIG. 2).

Heart H is a human heart that receives blood from and delivers blood to vasculature V. Heart H includes four chambers: right atrium RA, right ventricle RV, left atrium LA, and left ventricle LV.

The right side of heart H, including right atrium RA and right ventricle RV, receives deoxygenated blood from vasculature V and pumps the blood to the lungs. Blood flows into right atrium RA from superior vena cava SVC, inferior vena cava IVC, and coronary sinus CS.

A majority of the blood flows into right atrium RA from superior vena cava SVC and inferior vena cava IVC, which are offset from one another. Due to the offset of the major entry blood flows from superior vena cava SVC and inferior vena cava IVC, a natural flow vortex occurs in right atrium RA (a right-sided flow vortex). This allows a substantial portion of blood from right atrium RA to pass through right atrium RA and enter right ventricle RV by direct flow. The right-sided flow vortex in right atrium RA preserves kinetic energy and momentum of the major blood flows entering right atrium RA and allows a substantial portion of blood to naturally pass from right atrium RA to right ventricle RV without any contribution to flow needed from the pumping action of right atrium RA. With contraction, right atrium RA also pumps the residual portion of the entering blood not caught in the direct flow through tricuspid valve TV into right ventricle RV. The blood enters right ventricle RV and then flows through pulmonary valve PV into pulmonary artery PA. With preservation of direct inflow from right atrium RA, blood entering right ventricle RV also forms a natural flow vortex (a right-ventricular flow vortex) in right ventricle RV, which naturally re-directs blood entering right ventricle RV to pulmonary artery PA by direct flow without requiring right ventricle RV to perform substantial work of pumping blood. Residual blood that is not transported to pulmonary artery PA via pulmonary valve PV by direct flow is pumped by the contraction of right ventricle RV. The blood flows from pulmonary artery PA into smaller arteries that deliver the deoxygenated blood to the lungs via the pulmonary circulatory system. The lungs can then oxygenate the blood.

The left side of heart H, including left atrium LA and left ventricle LV, Receives the oxygenated blood from the lungs and provides blood flow to the body. Blood flows into left atrium LA from pulmonary veins PVS. The offset of the right and left pulmonary veins PVS also leads to the formation of a natural flow vortex in left atrium LA (left-sided flow vortex), which helps maintain momentum and minimize work as the blood traverses left atrium LA to mitral valve MV. Direct flow, as described above, and the pumping action of left atrium LA propels the blood through mitral valve MV into left ventricle LV. As the blood enters left ventricle LV, a natural flow vortex (a left-ventricular flow vortex) forms in left ventricle LV, which redirects flow naturally towards the left ventricular outflow of aortic valve AV so that it can be efficiently pumped by left ventricle LV through aortic valve AV into aorta AT. The blood flows from aorta AT into arteries that deliver the oxygenated blood to the body via the systemic circulatory system.

Blood is additionally received in right atrium RA from coronary sinus CS. Coronary sinus CS collects deoxygenated blood from the heart muscle and delivers it to right atrium RA. Thebesian valve BV is a semicircular fold of tissue at the opening of coronary sinus CS in right atrium RA. Coronary sinus CS is wrapped around heart H and runs in part along and beneath the floor of left atrium LA right above mitral valve MV, as shown in FIG. 2. Coronary sinus CS has an increasing diameter as it approaches right atrium RA. Coronary sinus CS also wraps around a portion of right atrium RA posteriorly before in enters right atrium RA via the ostium of coronary sinus CS lateral and posterior to an orifice of tricuspid valve TV, and medial to inferior vena cava IVC entry point. Due to its proximity to inferior vena cava IVC, blood entering right atrium RA from coronary sinus CS is naturally entrained into the larger inflow from inferior vena cava IVC forming the natural flow vortex (right-sided flow vortex) in right atrium RA, which naturally redirects the inflows towards tricuspid valve TV.

Inter-atrial septum IS and fossa ovalis FS are also shown in FIG. 2. Inter-atrial septum IS is the wall that separates right atrium RA from left atrium LA. Fossa ovalis FS is a depression in inter-atrial septum IS in right atrium RA. At birth, a congenital structure called a foramen ovale is positioned in inter-atrial septum IS. The foramen ovale is an opening in inter-atrial septum IS that closes shortly after birth to form fossa ovalis FS. The foramen ovale serves as a functional shunt in utero, allowing blood, primarily from inferior vena cava IVC and coronary sinus CS, to move from right atrium RA to left atrium LA to then be circulated through the body. This is necessary in utero, as the lungs are in a sack of fluid and do not oxygenate the blood. Rather, oxygenated blood is received from the mother. The oxygenated blood from the mother flows from the placenta into inferior vena cava IVC through the umbilical vein and enters the inferior vena cava IVC via a natural shunt called the ductus venosus. The oxygenated blood moves through inferior vena cava IVC to right atrium RA. The opening of inferior vena cava IVC in right atrium RA is positioned to direct the oxygenated blood through right atrium RA and then through a second natural shunt called foramen ovale into left atrium LA along with the entrained deoxygenated blood from coronary sinus CS. Left atrium LA can then pump the mixed oxygenated and deoxygenated blood into left ventricle LV, which pumps it to aorta AT and the systemic circulatory system. This allows the pulmonary circulatory system to be bypassed in utero. Some deoxygenated blood, primarily from superior vena cava SVC, is pumped through the right heart where it also bypasses the lungs and reenters aorta AT via a third natural shunt called the ductus arteriosus. Upon birth, respiration expands the lungs, blood begins to circulate through the lungs to be oxygenated, and the three natural shunts close. The closure of the foramen ovale forms fossa ovalis FS.

Shunt devices can be positioned in heart H to shunt blood between left atrium LA and right atrium RA. Left atrium LA has a higher pressure and lower compliance compared to right atrium RA, and right atrium RA has a lower pressure and higher compliance than left atrium LA. Left atrium LA can experience elevated pressure due to abnormal heart conditions. It has been hypothesized that patients with elevated pressure in left atrium LA may benefit from a reduction of pressure in left atrium LA. Shunt devices can be used in these patients to shunt blood from left atrium LA to right atrium RA to reduce the pressure of blood in left atrium LA, which reduces the systolic preload on left ventricle LV. Reducing pressure in left atrium LA further relieves back-pressure on the pulmonary circulation to reduce the risk of pulmonary edema. Reduction of back pressure on the pulmonary circulation also reduces pulmonary artery PA pressures, which can injure the small arteries leading to the lungs resulting in pulmonary hypertension. Increased pulmonary artery pressures can also lead to pressure overload of right ventricle RV, injuring right ventricle RV and potentially leading to right sided heart failure.

For example, shunt devices can be used to treat patients with heart failure (also known as congestive heart failure). The hearts of patients with heart failure do not pump blood as well as they should. Heart failure can affect the right side and/or the left side of the heart. Diastolic heart failure (also known as heart failure with preserved ejection fraction) refers to heart failure occurring when the left ventricle is stiff (having less compliance), which makes it hard to relax appropriately and fill with blood. This leads to increased end-diastolic pressure, which causes an elevation of pressure in left atrium LA. There are very few, if any, effective treatments available for diastolic heart failure. Other examples of abnormal heart conditions that cause elevated pressure in left atrium LA are systolic dysfunction of left ventricle LV and certain forms of congenital heart and valve disease.

Septal shunt devices (also called inter-atrial shunt devices or trans-septal shunt devices) are positioned in inter-atrial septum IS to shunt blood directly from left atrium LA to right atrium RA. Typically, septal shunt devices are positioned in fossa ovalis FS, as fossa ovalis FS is a thinner area of tissue in inter-atrial septum IS where the two atria share a common wall. If the pressure in right atrium RA exceeds the pressure in left atrium LA, septal shunt devices can allow blood to flow primarily from right atrium RA to left atrium LA. This causes a risk of paradoxical stroke (also known as paradoxical embolism), as emboli can move from right atrium RA to left atrium LA via the relatively short flow path of the shunt and then through left atrium LA into aorta AT and the systemic circulation as a result of physiologic conditions that may cause temporary bidirectional flow at different times in the cardiac cycle.

Shunt devices can also be left atrium to coronary sinus shunt devices that are positioned in a tissue wall between left atrium LA and coronary sinus CS where the two structures are in close approximation as coronary sinus CS passes through the atrio-ventricular groove that is covered by epicardium. Left atrium to coronary sinus shunt devices move blood from left atrium LA into coronary sinus CS, which then delivers the blood to right atrium RA via the ostium of coronary sinus CS, the natural orifice of coronary sinus CS, which may have thebesian valve BV. Coronary sinus CS is compliant and can quickly grow in response to increased volume with conditions such as drainage of the left subclavian vein to coronary sinus CS. Similarly, coronary sinus CS can act as an additional compliance chamber when using a left atrium to coronary sinus shunt device. Left atrium to coronary sinus shunt devices may further provide increased protections against paradoxical strokes by increasing the length of the flow path blood must traverse to get from right atrium RA to left atrium LA, as the blood would have to flow retrograde from right atrium RA through a significant distance in coronary sinus CS before entering left atrium LA. Further, left atrium to coronary sinus shunt devices also provide protection against significant right atrium RA to left atrium LA shunting of fully deoxygenated blood as it would have to flow retrograde from right atrium RA through coronary sinus CS for a substantial distance before entering left atrium LA.

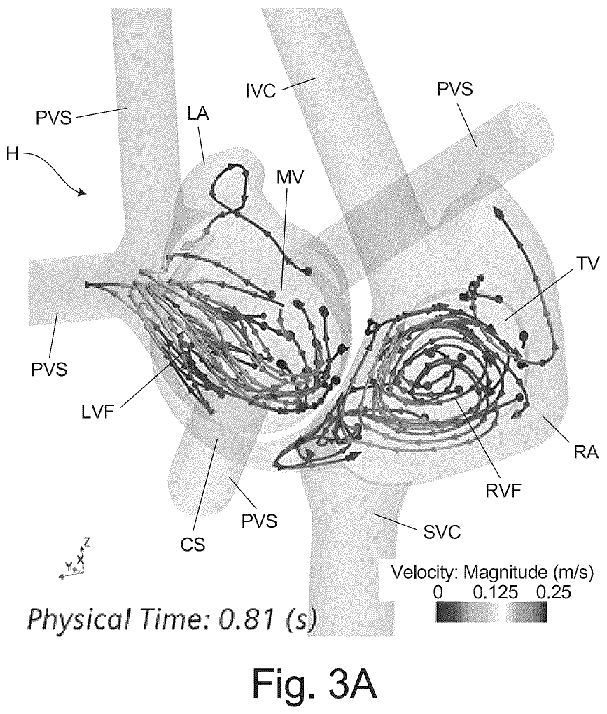

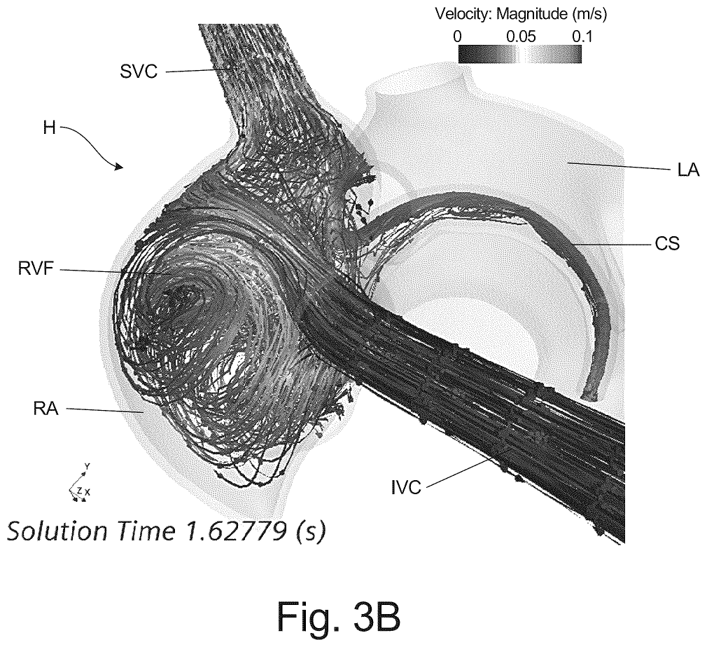

It has also been hypothesized that a left atrium to coronary sinus shunt device has a lesser disruption on the natural flow patterns of blood moving through left atrium LA, right atrium RA, and coronary sinus CS as compared to a traditional septal shunt device. Further, it is hypothesized that a left atrium to coronary sinus shunt device can enhance the natural vortical flow pattern of blood moving through right atrium RA, as the blood from coronary sinus CS is entrained into inferior vena cava IVC inflow. These flow patterns will be discussed below in greater detail with respect to FIGS. 3A-5B.

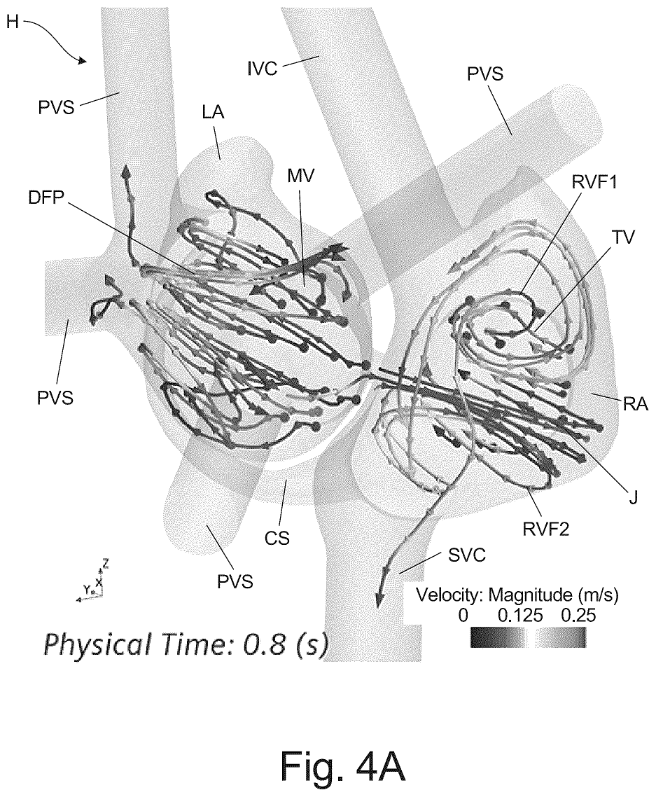

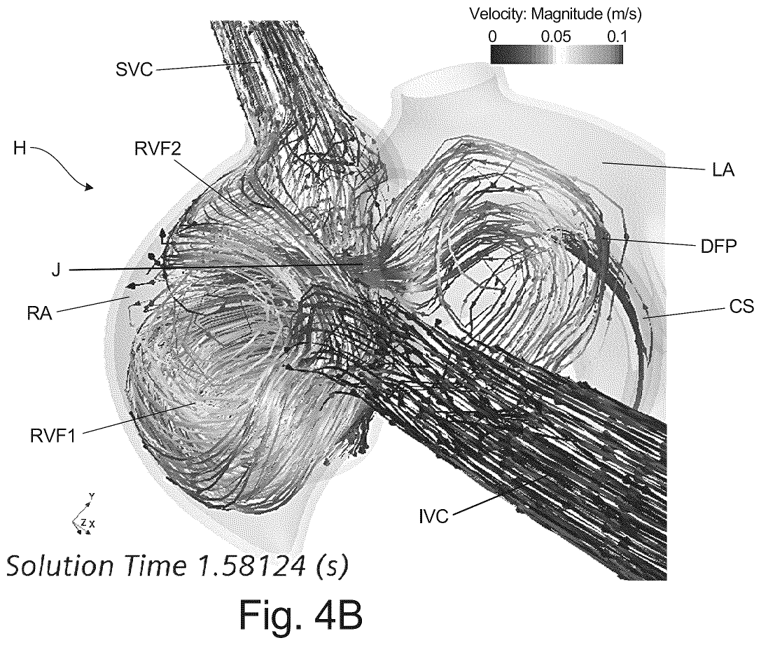

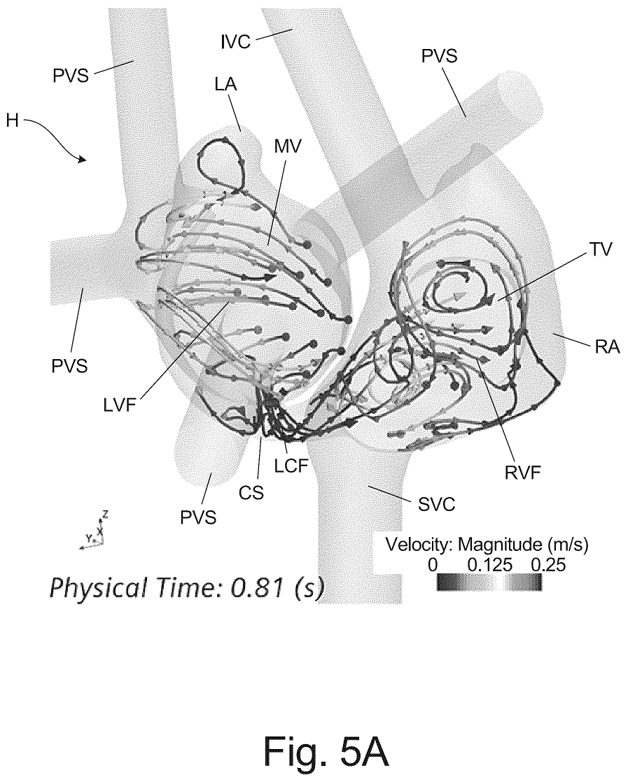

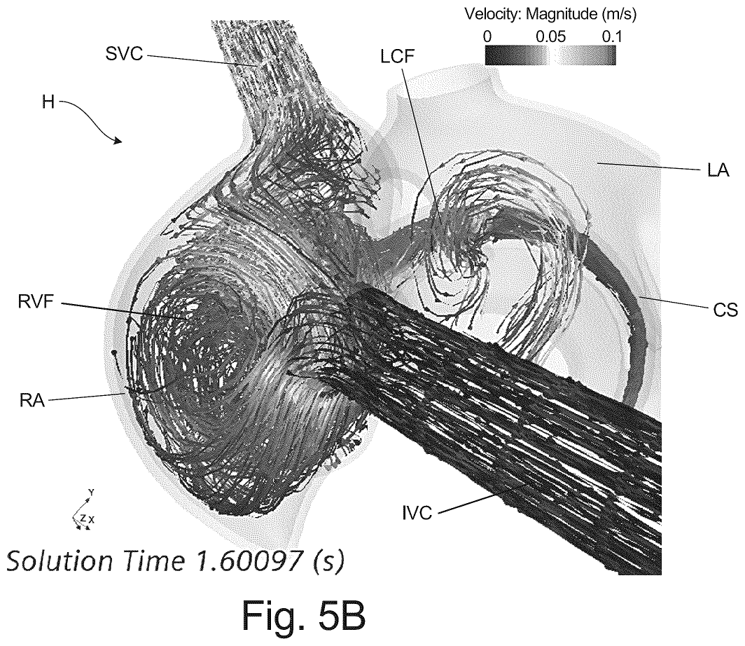

FIG. 3A is a first schematic diagram illustrating modeled hemodynamic flow patterns in heart H. FIG. 3B is a second schematic diagram illustrating modeled hemodynamic flow patterns in heart H. FIG. 4A is a first schematic diagram illustrating modeled hemodynamic flow patterns in heart H with a septal shunt device. FIG. 4B is a second schematic diagram illustrating modeled hemodynamic flow patterns in heart H with a septal shunt device. FIG. 5A is a schematic diagram illustrating modeled hemodynamic flow patterns in heart H with a left atrium to coronary sinus shunt device. FIG. 5B is a second schematic diagram illustrating modeled hemodynamic flow patterns in heart H with a left atrium to coronary sinus shunt device. FIGS. 3A-5B show heart H, right atrium RA, left atrium LA, superior vena cava SVC, inferior vena cava IVC, and coronary sinus CS. FIGS. 3A, 4A, and 5A show tricuspid valve TV, pulmonary veins PVS, and mitral valve MV.

FIGS. 3A-5B show modeled velocity stream lines representing hemodynamic flow patterns in heart H. FIGS. 3A, 4A, and 5A show heart H oriented with right atrium RA on a right side of the figures and left atrium LA on a left side of the figures. FIGS. 3A, 4A, and 5A are inferior views of heart H. FIGS. 3B, 4B, and 5B show heart H oriented with right atrium RA on a left side of the figures and left atrium LA on a right side of the figures. FIGS. 3B, 4B, and 5B are superior views of heart H.

Natural flow patterns of blood flow exist in heart H and help move blood through heart H and into the vasculature connected to heart H in a way that maximizes preservation of blood flow momentum and kinetic energy. The natural flow pattern for blood moving through arteries and veins is typically helical in nature (helical flow patterns). The natural flow pattern for blood moving through the chambers of heart H is typically vortical in nature (vortical flow patterns).

FIG. 3A shows modeled hemodynamic flow patterns that exist in right atrium RA and left atrium LA of heart H. FIG. 3B shows modeled hemodynamic flow patterns that exist in right atrium RA, superior vena cava SVC, inferior vena cava IVC, and coronary sinus CS. FIGS. 3A-3B represent natural flow patterns that are formed in heart H, including right atrium RA and left atrium LA, based on the offset inflows of blood into the chambers of heart H in addition to the anatomical structure of heart H. When looking at heart H from the right side (the right sagittal view), a clockwise right-sided flow vortex is formed in right atrium RA and a counter-clockwise left-sided flow vortex is formed in left atrium LA. The right-sided flow vortex in right atrium RA is the natural flow pattern of blood flow in right atrium RA. The left-sided flow vortex in left atrium LA is the natural flow pattern of blood flow in left atrium LA. The modeled hemodynamic flow patterns shown in FIGS. 3A-3B represent intra-cardiac flow patterns for a structurally normal heart.

Blood flows enter the right atrium RA from superior vena cava SVC, inferior vena cava IVC, and coronary sinus CS. The superior vena cava opening and the inferior vena cava opening in right atrium RA are offset so that the blood flowing into right atrium RA from superior vena cava SVC and inferior vena cava IVC do not collide with each other. Due to its orientation and physical proximity, coronary sinus CS flow is entrained into inferior vena cava IVC flow. The blood flowing through superior vena cava SVC and inferior vena cava IVC has a helical flow pattern. A majority of the blood in right atrium RA enters right atrium RA through inferior vena cava IVC, and the blood flowing from inferior vena cava IVC into right atrium RA is pointed towards the top of right atrium RA. The helical flow pattern of the blood flowing into right atrium RA from inferior vena cava IVC helps to form a clockwise right-sided flow vortex in right atrium RA (when looking at the heart from the right side). The flow of blood entering right atrium RA from superior vena cava SVC will flow along the inter-atrial septum and towards tricuspid valve TV. The helical flow pattern of the blood flowing from superior vena cava SVC into right atrium RA helps the flow of blood naturally join with the clockwise right-sided flow vortex formed in right atrium RA by the flow of blood from inferior vena cava IVC, which is joined by coronary sinus CS flow. A small amount of blood flows into right atrium RA from coronary sinus CS. The flow flowing through coronary sinus CS will have a helical flow pattern. The helical flow pattern of the blood exiting coronary sinus CS will naturally join with inferior vena cava IVC flow and the right-sided flow vortex in right atrium RA. The right-sided flow vortex in right atrium RA is shown with velocity stream lines labeled RVF in FIGS. 3A-3B.

The right-sided flow vortex formed in right atrium RA helps the blood flow through right atrium RA, through tricuspid valve TV, into the right ventricle, through the pulmonary valve, and into the pulmonary artery. The right heart is an inefficient pump and can act more like a conduit. The right-sided flow vortex formed in the right heart helps to preserve kinetic energy and the momentum of blood flow as it moves from superior vena cava SVC and inferior vena cava IVC (the Vena Cavac) through the right heart and into the pulmonary artery, even with minimal to no pumping being provided by the right heart. This is especially important for maintaining right heart output, which must match left heart output, during periods of high output and heart rates during exercise. The right-sided flow vortex formed in right atrium RA helps to move the blood from right atrium RA through tricuspid valve TV and into the right ventricle with minimal loss of momentum and kinetic energy. The blood shoots from right atrium RA through the right ventricle, out the right ventricular outflow tract, through the pulmonary valve, and into the pulmonary artery. Approximately 50% of the blood will flow into the pulmonary artery without any pumping required by the right heart because of the right-sided flow vortices of right atrium RA and right ventricle RV and anatomical constraints of the right heart. Right heart contraction enhances the flow of residual blood through the right heart.

Blood flows into left atrium LA from pulmonary veins PVS. There are four pulmonary veins PVS that flow into left atrium LA. The blood flowing through pulmonary veins PVS has a helical flow pattern. The offset of helical flow of the blood flowing from pulmonary veins PVS into left atrium LA helps to form a counter-clockwise left-sided flow vortex (when looking at the heart from the right side) in left atrium LA. The left-sided flow vortex in left atrium LA directs flow towards mitral valve MV. The left-sided flow vortex in left atrium LA is shown with velocity stream lines labeled LVF in FIG. 3A.

It is hypothesized that if the intra-cardiac blood flow patterns in heart H (including right-sided flow vortex in right atrium RA and left-sided flow vortex in left atrium LA) are disrupted, blood flow from superior vena cava SVC and inferior vena cava IVC (the Vena Cavac), through right atrium RA, through the right ventricle, and into the pulmonary artery, and blood flow from the pulmonary veins, through the left atrium LA, through the left ventricle, and into the aorta become less efficient and place increased mechanical workloads on the respective ventricles. This is especially important in already failing hearts, where the ability to increase the workload of the heart muscle is impaired. Disruptions in the intra-cardiac blood flow patterns in heart H (including right-sided flow vortex in right atrium RA and left-sided flow vortex in left atrium LA) can happen for a variety of reasons. For example, the anatomy of heart H can change as patients age. This can affect the offset between the opening of superior vena cava SVC and the opening of inferior vena cava IVC. The blood flow entering right atrium RA from superior vena cava SVC and the blood flow entering right atrium RA from inferior vena cava IVC can collide as the anatomy of heart H changes, which disrupts the natural formation of the right-sided flow vortex in right atrium RA. In another example, right atrium RA can be enlarged in patients with heart failure with or without atrial fibrillation. The enlargement of right atrium RA can also disrupt the right-sided flow vortex formed in right atrium RA. Similarly, left atrium LA can be enlarged in patients with heart failure with or without atrial fibrillation. The enlargement of left atrium LA can disrupt the left-sided flow vortex formed in left atrium LA. Additionally, patients with a patent foramen ovale (a natural inter-atrial septal shunt) or a secundum atrial septal defect due to failure of the patent foramen ovale to fully close may not have the expected intra-cardiac blood flow patterns (including right-sided flow vortex in right atrium RA and left-sided flow vortex in left atrium LA), including the expected flow vortexes created during atrial filling. Closure of a secundum atrial septal defect with altered right atrial non-single vortex flow patterns has been shown to revert to a dominant single vortical flow pattern after the atrial septal defect is occluded.

When the right-sided flow vortex in right atrium RA changes, momentum and energy of the blood flow are lost, and the right heart needs to pump harder to move the blood from right atrium RA into the right ventricle and the pulmonary artery. This is due to the right-sided flow vortex contributing less to the movement of blood through the right heart. Similarly, when the left-sided flow vortex in left atrium LA changes, the left heart needs to pump harder to move the blood from left atrium LA into the left ventricle and the aorta. This is due to the left-sided flow vortex contributing less to the movement of blood through the left heart. Further, as the intra-cardiac flow patterns in heart H (including right-sided flow vortex in right atrium RA and left-sided flow vortex in left atrium LA) change due to age or disease, areas of turbulence can be created in the flow patterns of heart H and there can be a loss of fluid dynamics leading to inefficiencies that could lead to diminished flow. This can increase the susceptibility of the right heart and/or the left heart to fail (the inability to pump enough blood to meet the body's oxygen demands), as heart H has to do more work to move the same amount of blood through heart H. More work is needed to recreate the lost momentum naturally preserved by the intra-cardiac flow patterns in heart H (including right-sided flow vortex in right atrium RA and left-sided flow vortex in left atrium LA), putting additional strain on heart H.

Changes in intra-cardiac flow patterns change intra-cardiac energetics. Heart H is uniquely designed to maximize efficiency by preserving the kinetic energy and momentum of blood flow, thus minimizing the work needed to propagate the blood flow into the chambers, between the chambers, and out of the chambers. Anything that disrupts the intra-cardiac flow patterns in heart H (including right-sided flow vortex in right atrium RA and left-sided flow vortex in left atrium LA) can reduce the efficiency of the energetics of heart H due to a loss of potential energy, which makes it more difficult for heart H to do its job of propagating blood into, between, and out of the chambers. Anything that disrupts the intra-cardiac flow patterns through heart H (including right-sided flow vortex in right atrium RA and left-sided flow vortex in left atrium LA) can increase the amount of work heart H has to do, prolong transit times through heart H, and makes it more difficult for heart H to eject blood. This is especially problematic for people experiencing heart failure, as the heart failure can be exacerbated due to disruptions in the intra-cardiac flow patterns through heart H (including right-sided flow vortex in right atrium RA and left-sided flow vortex in left atrium LA).

FIG. 4A shows modeled hemodynamic flow patterns that exist in right atrium RA and left atrium LA of heart H when a septal shunt device is positioned between right atrium RA and left atrium LA. FIG. 4B shows modeled hemodynamic flow patterns that exist in right atrium RA, superior vena cava SVC, inferior vena cava IVC, coronary sinus CS, and left atrium LA when a septal shunt device is positioned between right atrium RA and left atrium LA. A septal shunt device has been modeled in the inter-atrial septum between right atrium RA and left atrium LA in the schematic shown in FIGS. 4A-4B to allow blood to shunt directly from left atrium LA to right atrium RA.

As shown in FIGS. 4A-4B, when a septal shunt device is positioned in the inter-atrial septum between right atrium RA and left atrium LA, blood jets from left atrium LA into and across right atrium RA. The jet of blood is shown with velocity stream lines labeled J in FIGS. 4A-4B. The jet of blood in right atrium RA disrupts the right-sided flow vortex in right atrium RA. When the blood jets across right atrium RA, two separate flow vortexes are formed in right atrium RA. The first flow vortex is shown with velocity stream lines labeled RVF1, and the second flow vortex is shown with velocity stream lines labeled RVF2 in FIGS. 4A-4B. There is also a disruption of the left-sided flow vortex in left atrium LA. The septal shunt device is not aligned with the left-sided flow vortex in left atrium LA, but the pressure difference between right atrium RA and left atrium LA causes the blood in left atrium LA to move out of the left-sided flow vortex and through the septal shunt device into right atrium RA. The disrupted left-sided flow vortex in left atrium LA is shown with velocity stream lines labeled DFP in FIGS. 4A-4B. This disruption of the right-sided flow vortex in right atrium RA and the left-sided flow vortex in left atrium LA can also lead to loss of right ventricle RV and left ventricle LV vortex formations and will cause heart H to have to work harder to pump blood through their respective ventricles and can lead to the development or worsening of heart failure over time.

Specifically, when looking at the right heart, a septal shunt device introduces a significant disruption to the right-sided flow vortex in right atrium RA as the blood jets across right atrium RA. It is hypothesized that the disruption to the right-sided flow vortex in right atrium RA can cause or exacerbate right heart failure. Disruption of the right-sided flow vortex in right atrium RA means that the momentum and kinetic energy of blood naturally or efficiently flowing from right atrium RA into the right ventricle and the pulmonary artery is lost. In order to move the blood from right atrium RA into the right ventricle and the pulmonary artery, the right heart has to work harder to pump the blood. This increased work required by the right heart can cause or exacerbate right heart failure and places a severe load on the less efficient right heart during periods of exercise, where heart rates are high and diastolic filling periods are short.

FIG. 5A shows modeled hemodynamic flow patterns that exist in right atrium RA and left atrium LA of heart H when a left atrium to coronary sinus shunt device is positioned in a tissue wall between left atrium LA and coronary sinus CS. FIG. 5B shows modeled hemodynamic flow patterns that exist in right atrium RA, superior vena cava SVC, inferior vena cava IVC, coronary sinus CS, and left atrium LA when a left atrium to coronary sinus shunt device is positioned in a tissue wall between left atrium LA and coronary sinus CS. A shunt device has been modeled in the tissue wall between left atrium LA and coronary sinus CS in the schematics shown in FIGS. 5A-5B to allow blood to shunt from left atrium LA to coronary sinus CS, which then delivers the blood to right atrium RA.

As shown in FIGS. 5A-5B, when a left atrium to coronary sinus shunt device is positioned in the tissue wall between left atrium LA and coronary sinus CS, blood moves from left atrium LA into coronary sinus CS. The blood moving from left atrium LA to coronary sinus CS is shown with velocity stream lines labeled LCF in FIGS. 5A-5B. The blood moving from left atrium LA to coronary sinus CS minimizes disruption to the flow of blood in left atrium LA and coronary sinus CS. The blood moves from left atrium LA to coronary sinus CS at a high velocity due to the pressure difference between left atrium LA and coronary sinus CS. Positioning a shunt device in the tissue wall between left atrium LA and coronary sinus CS close to the mitral annulus positions the shunt device to guide blood from the left-sided flow vortex in left atrium LA through the shunt device to join with the flow pattern in coronary sinus CS. As blood flows in the left-sided flow vortex in left atrium LA (shown with velocity stream lines labeled LVF in FIG. 3C), it flows against the tissue wall where the left atrium to coronary sinus shunt device is placed and will naturally flow through the shunt device. This minimizes the disruption to the left-sided flow vortex in left atrium LA. Further, the blood that flows through the left atrium to coronary sinus shunt device joins into the helical flow of blood flowing through coronary sinus CS. This minimizes disruptions to the flow of blood through coronary sinus CS. The pressure of the blood in coronary sinus CS will increase due to the increase in volume of blood in coronary sinus CS. Further, coronary sinus CS can dilate due to the increased volume of blood in coronary sinus CS.

As blood normally flows from coronary sinus CS into right atrium RA where it is entrained into the flow path of inferior vena cava IVC, there is minimal to no disruption of the right-sided flow vortex in right atrium RA (shown with velocity stream lines labeled RVF in FIGS. 5A-5B). The modeled flow patterns shown in FIGS. 5A-5B show less disruption of the right-sided flow vortex in right atrium RA as compared to the modeled flow patterns when using a septal shunt device as shown in FIGS. 4A-4B. The minimal to no disruption of the right-sided flow vortex in right atrium RA with a left atrium to coronary sinus shunt reduces energy loss in right atrium RA, thus requiring less work from the right heart to pump blood through the right atrium to the right ventricle and the pulmonary artery. A left atrium to coronary sinus shunt device can provide fluidic and dynamic advantages that have the potential to avoid disruptions in asymmetric redirection of streaming blood flow through the right heart. Further, it can preserve the right-sided flow vortex that arises in right atrium RA during the atrial filling stage, which may transport the momentum efficiently to reduce energy loss.

It is hypothesized that the increased velocity of the flow of blood entering right atrium RA from coronary sinus CS when using a left atrium to coronary sinus shunt device enhances and/or augments the right-sided flow vortex formed in right atrium RA. The increased velocity of the flow of blood entering right atrium RA from coronary sinus CS when using a left atrium to coronary sinus shunt device can help to reestablish or reinforce the right-sided flow vortex in right atrium RA for patients who have lost their right-sided flow vortex due to age and/or disease. The flow from coronary sinus CS exits the ostium of coronary sinus CS, flows upwards in right atrium RA and as it is entrained into the inferior vena cava flow, may augment and/or reestablish the vortical flow pattern in right atrium RA. Alternatively, the now enhanced flow from coronary sinus CS, which in the presence of a shunt device increases to 20-40% of cardiac output, may direct blood through tricuspid valve TV in a path that preferentially shoots blood out the right ventricle outflow tract to the pulmonary artery. A left atrium to coronary sinus shunt device can preserve, and may even enhance, the momentum of inflowing blood streams being redirected towards atrio-ventricular valves of heart H. Further, it can preserve, and may even enhance, the normal exit of blood flow from right atrium RA to the right ventricle, which promotes change in flow direction at a ventricular level such that recoil away from ejected blood is in a direction that can enhance rather than inhibit ventriculo-atrial coupling.

A left atrium to coronary sinus shunt device takes advantage of normal flow paths in heart H and minimizes dissipative interaction between entering, recirculating, and outflowing blood streams. Recirculating flows and vortices are characteristic flow features in between cardiac chambers, which play a crucial role in momentum transfer and irreversible energy loss. A left atrium to coronary sinus shunt device preserves the natural intra-atrial and intra-ventricular flow structures of the healthy human heart that are optimal for minimizing energy dissipation. An increase in energy dissipation due to the break of the natural flow structure may lead to an increase in the energy that is needed to be generated by myocardial muscle to eject the blood into the circulation (increases myocardial work in an already pressure overloaded ventricle).

A left atrium to coronary sinus shunt device does not interfere with the reciprocating, sling-like “morphodynamic” mode of action that comes into effect when heart rate and output increases during exercise. It is hypothesized that a left atrium to coronary sinus shunt device has potential functional advantages that could gain importance as flow velocities, heart rate and rates of change of momentum increase with exertion. A left atrium to coronary sinus shunt device does not detract from the ability of the looped heart to deliver enhanced output during strenuous exertion. Rather it will enhance responses to exercise, as the looped heart is able to function “morphodynamically,” redirecting and slinging blood through its sinuous curvatures with minimal dissipation of energy and with dynamically enhanced reciprocation of atrial and ventricular function.

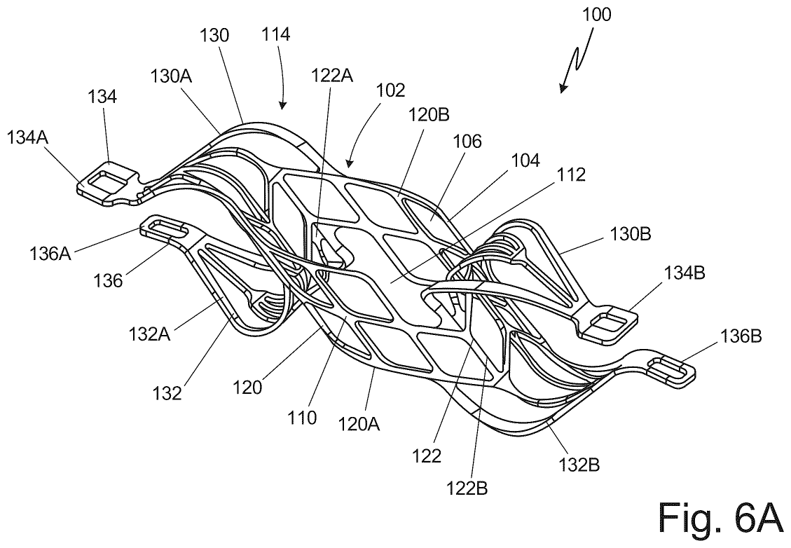

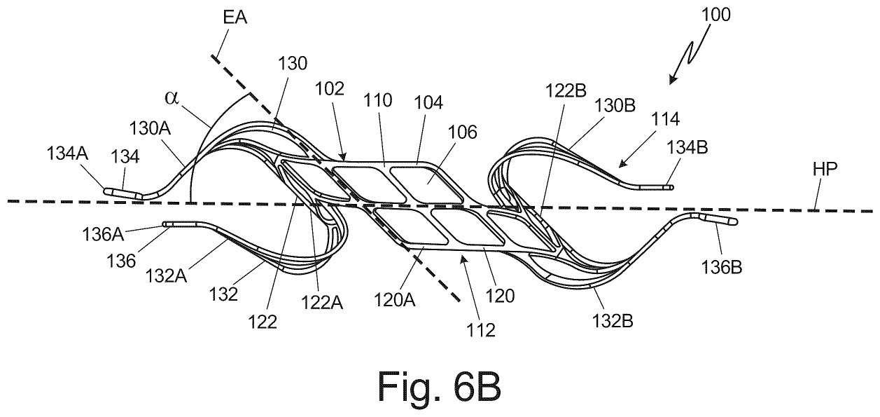

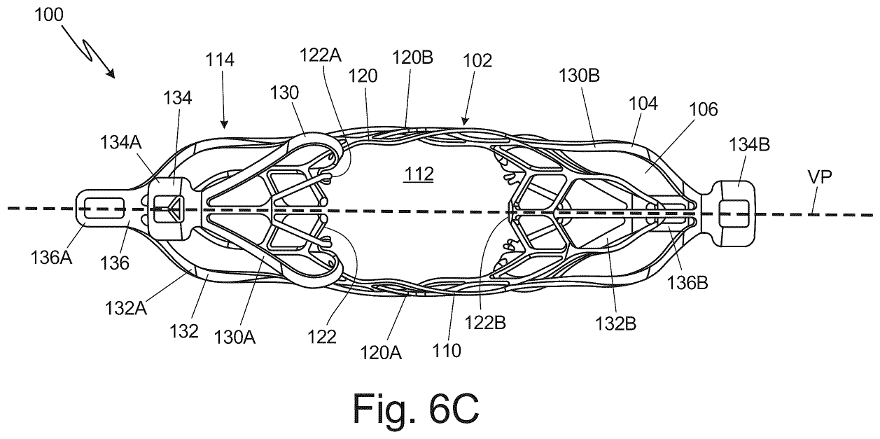

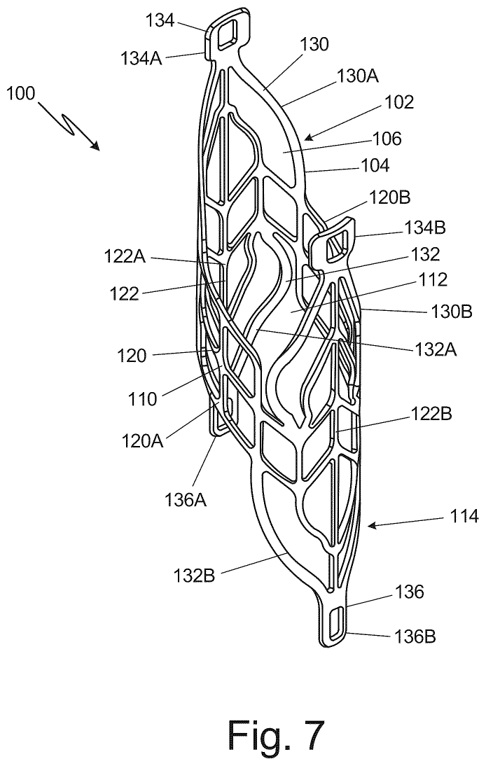

FIG. 6A is a perspective view of shunt device 100. FIG. 6B is a side view of shunt device 100. FIG. 6C is a bottom view of shunt device 100. FIG. 7 is a perspective view of shunt device 100 in a collapsed configuration. FIGS. 6A, 6B, 6C and 7 will be discussed together. Shunt device 100 includes body 102, which is formed of struts 104 and openings 106. Body 102 includes central flow tube 110, flow path 112, and arms 114. Central flow tube 110 has side walls 120 (including side wall 120A and side wall 120B) and end walls 122 (including end wall 122A and end wall 122B). Arms 114 include distal arms 130 (including distal arm 130A and distal arm 130B) and proximal arms 132 (including proximal arm 132A and proximal arm 132B). Distal arms 130 have terminal ends 134 (including terminal end 134A and terminal end 134B). Proximal arms 132 have terminal ends 136 (including terminal end 136A and terminal end 136B). FIG. 6B further shows horizontal reference plane HP, end wall axis EA, and angle α. FIG. 6C further shows vertical reference plane VP.

Shunt device 100 is shown in an expanded configuration in FIGS. 6A-6C. Shunt device 100 is formed of a super-elastic material that is capable of being compressed into a catheter for delivery into the body. Shunt device 100 is shown in a compressed configuration in FIG. 7. Upon delivery into the body, shunt device 100 will expand back to its relaxed, or expanded, shape. Shunt device 100 has body 102 that is formed of interconnected struts 104. Openings 106 in body 102 are defined by struts 104. Body 102 of shunt device 100 is formed of struts 104 to increase the flexibility of shunt device 100 to enable it to be compressed and expanded. Shunt device 100 can be sterilized before being delivered into the body.

Body 102 includes central flow tube 110 that forms a center portion of shunt device 100. Central flow tube 110 is tubular in cross-section but is formed of struts 104 and openings 106. Central flow tube 110 can be positioned in a puncture in a tissue wall and holds the tissue wall open. Flow path 112 is an opening extending through central flow tube 110. Flow path 112 is the path through which blood flows through shunt device 100. Arms 114 extend from central flow tube 110. Arms 114 extend outward from central flow tube 110 when shunt device 100 is in an expanded configuration. Arms 114 hold shunt device 100 in position in the tissue wall when shunt device 100 is implanted in the body.

When shunt device 100 is implanted in the tissue wall between the left atrium and the coronary sinus, central flow tube 110 holds the tissue wall open so blood can flow from the left atrium to the coronary sinus through flow path 112. Struts 104 of central flow tube 110 form a cage of sorts that is sufficient to hold the tissue wall open around central flow tube 110. Central flow tube 110 is designed to have a thickness that approximates the thickness of the tissue wall between the left atrium and the coronary sinus.

Central flow tube 110 has side walls 120 and end walls 122. Side wall 120A and side wall 120B form opposing sides of central flow tube 110. End wall 122A and end wall 122B form opposing ends of central flow tube 110. End wall 122A and end wall 122B each extend between and connect to side wall 120A and side wall 120B to form a circular opening that defines flow path 112. Struts 104 of central flow tube 110 define generally parallelogram-shaped openings 106 in central flow tube 110. Struts 104 of side walls 120 form an array of parallelogram-shaped openings 106 in side walls 120. Side walls 120 and end walls 122 form a tubular lattice for central flow tube 110.

As shown in FIG. 6B, central flow tube 110 is angled with respect to horizontal reference plane HP extending through shunt device 100. Horizontal reference plane HP lies generally in the plane of the tissue wall immediately adjacent to shunt device 100 when shunt device 100 is implanted. End walls 122 are angled with respect to horizontal reference plane HP. As shown in FIG. 6B, end walls 122 extend along end wall axis EA that extends at angle α with respect to horizontal reference plane HP. Angle α can be between 15° and 90°. Alternatively, angle α can be between 30° and 75°. Alternatively, angle α can be between 60° and 65°.

Arms 114 of shunt device 100 include two distal arms 130 and two proximal arms 132. Arms 114 extend outward from end walls 122 of central flow tube 110 when shunt device 100 is in an expanded configuration. Distal arm 130A is connected to and extends away from end wall 122A, and distal arm 130B is connected to and extends away from end wall 122B. Proximal arm 132A is connected to and extends away from end wall 122A, and proximal arm 132B is connected to and extends away from end wall 122B. When shunt device 100 is implanted in the tissue wall between the left atrium and the coronary sinus, distal arms 130 will be positioned in the left atrium and proximal arms 132 will be positioned in the coronary sinus.

Distal arms 130 and proximal arms 132 curl outward from end walls 122. As shown in FIG. 6C, distal arm 130A and distal arm 130B extend outwards from central flow tube 110 in opposite directions parallel to vertical reference plane VP. Distal arm 130A has a longer length than distal arm 130B. Proximal arm 132A and proximal arm 132B extend outwards from central flow tube 110 in opposite directions parallel to vertical reference plane VP. Proximal arm 132A has a shorter length than proximal arm 132B. Distal arm 130A has generally the same length and shape as proximal arm 132B, and distal arm 130B has generally the same length and shape as proximal arm 132A. As such, shunt device 100 is inversely symmetrical across horizontal reference plane HP, as shown in FIG. 6B.

Shunt device 100 is generally elongated longitudinally but is relatively narrow laterally. Stated another way, distal arms 130 and proximal arms 132 are not annular or circular, but rather extend outward generally in only one plane. As shown in FIG. 6B, shunt device 100 has a generally H-shape when viewing a side of shunt device 100. The elongated shape of shunt device 100 means that when compressed it elongates along a line, as shown in FIG. 7, so as to better fit within a catheter.

Distal arms 130 each have terminal ends 134. Specifically, distal arm 130A has terminal end 134A, and distal end 130B has terminal end 134B. Proximal arms 132 each have terminal ends 136. Specifically, proximal arm 132A has terminal end 136A, and proximal arm 132B has terminal end 136B. Terminal ends 134 of distal arms 130 and terminal ends 136 of proximal arms 132 converge towards one another. Distal arms 130 and proximal arms 132 form two pairs of arms. Distal arm 130A and proximal arm 132A form a first pair of arms extending outward from a first side of central flow tube 110, and terminal end 134A of distal arm 130A converges towards terminal end 136A of proximal arm 132A. Distal arm 130B and proximal arm 132B form a second pair of arms extending outward from a second side of central flow tube 110, and terminal end 134B of distal arm 130B converges towards terminal end 136B of proximal arm 132B. The gap between terminal ends 134 and terminal ends 136 is sized to be slightly smaller than an approximate thickness of the tissue wall between the left atrium and the coronary sinus. This allows distal arms 130 and proximal arms 132 to flex outwards and grip the tissue wall when implanted to help hold shunt device 100 in place in the tissue wall. Terminal ends 134 of distal arms 130 and terminal ends 136 of proximal arms 132 can also have openings that are configured to engage a delivery tool to facilitate implantation of shunt device 100, for example actuating rods of a delivery tool.

When implanted in the tissue wall, distal arms 130 and proximal arms 132 are designed such that the projection of distal arms 130 and proximal arms 132 into the left atrium and the coronary sinus, respectively, is minimized. This minimizes the disruption of the natural flow patterns in the left atrium and the coronary sinus. Shunt device 100 can also be designed so that the profile of proximal arms 132 projecting into the coronary sinus is lower than the profile of distal arms 130 projecting into the left atrium to minimize disruption of the natural blood flow through the coronary sinus.

Shunt device 100 and other examples of shunt devices are described in further detail in U.S. Pat. No. 9,789,294, filed on Oct. 6, 2016, issued on Oct. 17, 2017, and entitled “Expandable Cardiac Shunt,” the disclosure of which is incorporated by reference in its entirety. Shunt device 100 can be implanted in a tissue wall using a catheter-based method know in the art, for example as described in U.S. Pat. No. 9,789,294.

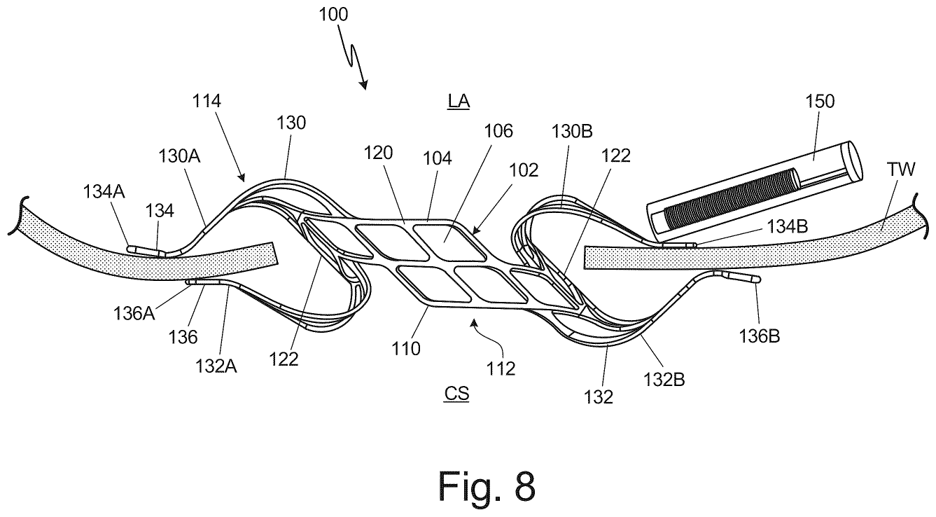

FIG. 8 is a side view of shunt device 100 with sensor 150 and anchored to tissue wall TW. Shunt device 100 includes body 102, which is formed of struts 104 and openings 106. Body 102 includes central flow tube 110, flow path 112, and arms 114. Central flow tube 110 has side walls 120 and end walls 122. Arms 114 include distal arms 130 (including distal arm 130A and distal arm 130B) and proximal arms 132 (including proximal arm 132A and proximal arm 132B). Distal arms 130 have terminal ends 134 (including terminal end 134A and terminal end 134B). Proximal arms 132 have terminal ends 136 (including terminal end 136A and terminal end 136B). FIG. 8 further shows sensor 150, tissue wall TW, left atrium LA, and coronary sinus CS.

Shunt device 100 is described above in reference to FIGS. 6A-7. Shunt device 100 as shown in FIG. 8 further includes sensor 150. Shunt device 100 is shown implanted in tissue wall TW. In the example shown in FIG. 8, sensor 150 will be positioned in left atrium LA when shunt device 100 is implanted in tissue wall TW. Sensor 150 is attached to distal arm 130B of shunt device 100 in the example shown in FIG. 8 but can be attached to distal arm 130A in alternate examples. In further examples, sensor 150 can be attached to proximal arm 132A or proximal arm 132B to be positioned in coronary sinus CS. Alternatively, an additional sensor can be included on shunt device 100 to position a sensor in both left atrium LA and coronary sinus CS. Sensor 150 can be integrally formed with shunt device 100 or attached to shunt device 100 using any suitable mechanism.

Sensor 150 can be a pressure sensor to sense a pressure in the left atrium. In other examples, sensor 150 can be any sensor to measure a parameter in the left atrium. Sensor 150 can include a transducer, control circuitry, and an antenna in one example. The transducer, for example a pressure transducer, is configured to sense a signal from the left atrium. The transducer can communicate the signal to the control circuitry. The control circuitry can process the signal from the transducer or communicate the signal from the transducer to a remote device outside of the body using the antenna. Sensor 150 can include alternate or additional components in other examples. Further, the components of sensor 150 can be held in a sensor housing that is hermetically sealed.

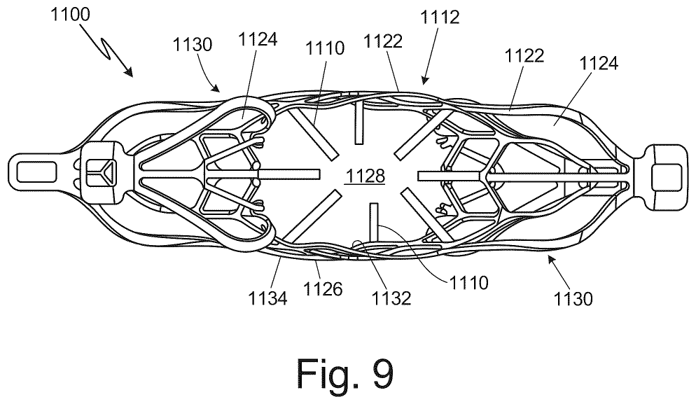

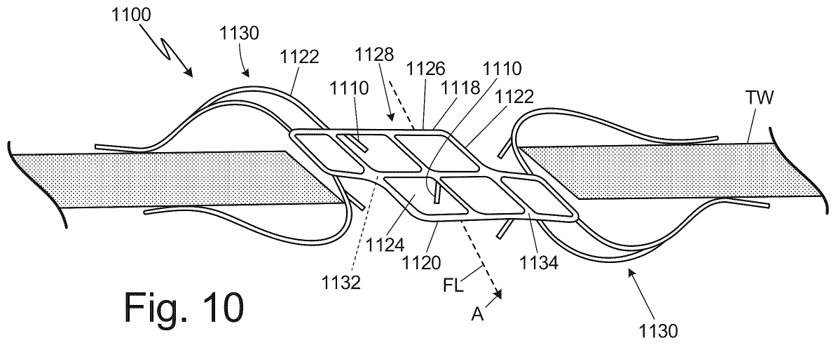

FIGS. 9-10 will be described together. FIG. 9 is a bottom view of shunt device 1100 including fin-type flow conditioners 1110. FIG. 10 is a side view of shunt device 1100 anchored to tissue wall TW and including fin-type flow conditioners 1110. FIGS. 9-10 show shunt device 1100 including fin-type flow conditioners 1110, body 1112, inflow end 1118 (shown in FIG. 10), and outflow end 1120 (shown in FIG. 10). Body 1112 is formed of struts 1122 and openings 1124. Body 1112 includes central flow tube 1126, flow path 1128, and arms 1130, and defines flow axis FL (shown in FIG. 10). Central flow tube 1126 includes inner surface 1132 and outer surface 1134. FIG. 10 also shows tissue wall TW.

Shunt device 1100 has a similar structure and function to shunt device 100 described above in reference to FIGS. 6A-8, except shunt device 1100 additionally includes fin-type flow conditioners 1110.

Shunt device 1100 can be described with reference to inflow end 1118 and outflow end 1120. Central flow tube 1126 of shunt device 1100 extends between inflow end 1118 and outflow end 1120. Inflow end 1118 can be an end of device 1100 that is relatively upstream of outflow end 1120 with respect to a flow of blood along flow axis FL, as represented by arrow A in FIG. 10, when device 100 is implanted in tissue wall TW (such as the inter-atrial septum or the tissue wall between the left atrium and the coronary sinus). Accordingly, outflow end 1120 is an end of device 1100 that is relatively downstream of inflow end 1118 with respect to a flow of blood along flow axis FL, as represented by arrow A in FIG. 10, when device 1100 is implanted in tissue wall TW (such as the inter-atrial septum or the tissue wall between the left atrium and the coronary sinus). In examples where device 1100 is a left atrium to coronary sinus shunt, inflow end 1118 can be facing the left atrium and outflow end 1120 can be facing the coronary sinus. In other examples where device 1100 is a septal shunt positioned between the left atrium and the right atrium, inflow end 1118 can be facing the left atrium and outflow end 1120 can be facing the right atrium. Although inflow end 1118 is defined as being relatively upstream of outflow end 1120, it should be understood that other actual positions of inflow end 1118 or outflow end 1120 are possible depending on the location where device 1100 is implanted.

Central flow tube 1126 of shunt device 1100 can also be described with reference to inner surface 1132 and outer surface 1134. Central flow tube 1126 has inner surface 1132 and outer surface 1134. Inner surface 1132 can be a radially inner surface of central flow tube 1126 with respect to flow axis FL therethrough. Outer surface 1134 can be a radially outer surface of central flow tube 1126 with respect to flow axis FL therethrough. Openings 1124 in body 1112 at central flow tube 1126 extend from inner surface 1132 to outer surface 1134. Flow path 1128 through central flow tube 1126 is defined by inner surface 1132. More specifically, flow axis FL is a central longitudinal axis through central flow tube 1126 of device 1100 along which blood flows as it passes through device 1100 in the direction indicated by the arrow A in FIG. 10.

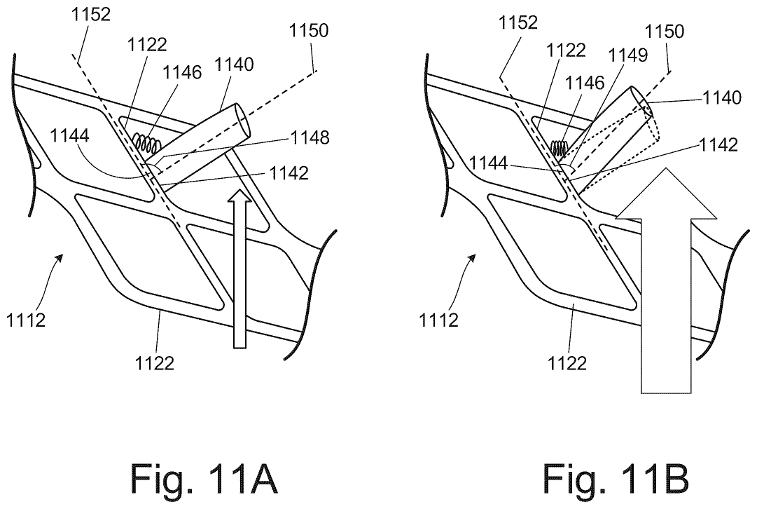

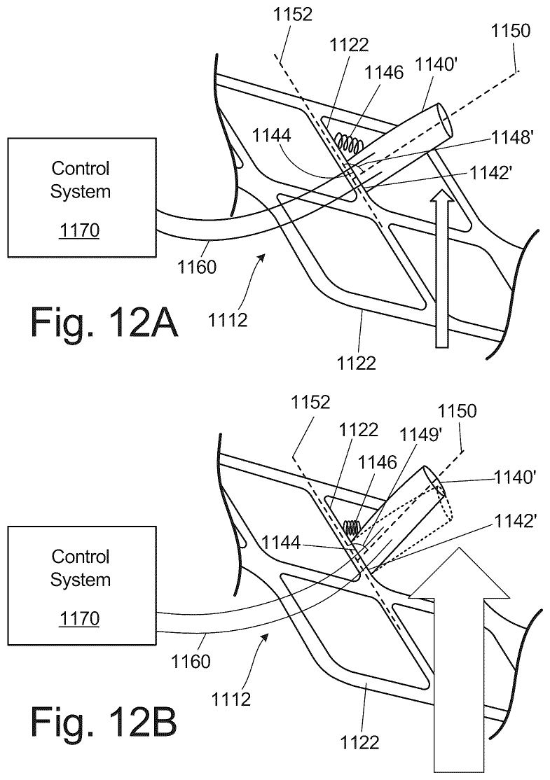

Flow conditioners 1110 are fins or fin-type flow conditioners. Each individual one of flow conditioners 1110 can also be referred to as a flow conditioner feature. Flow conditioners 1110 are elongated projections from body 1112. More specifically, flow conditioners 1110 are connected to body 1112 at corresponding ones of struts 1122. Flow conditioners 1110 can be connected to body 1112 at central flow tube 1126 and/or at ones of arms 1130. Flow conditioners 1110 are attached by an attachment mechanism (as described in greater detail with reference to FIGS. 11A-11B below) or monolithically formed with a portion of frame 112 (as described in greater detail with reference to FIGS. 12A-12B below).

In general, flow conditioners 1110 can take a number of different forms (i.e., shapes, sizes, etc.). In some examples, flow conditioners 1110 can be airfoils. Flow conditioners 1110 can have a symmetrical or asymmetrical and regular or irregular shape and can have variable geometries. Physical dimensions (e.g., length, width, shape, cross-sectional shape, etc.) of flow conditioners 1110 can be configured to prevent flow conditioners 1110 from interfering with other parts of device 1100 or adjacent tissue walls (e.g., tissue wall TW). In other examples, components of device 1100 can be designed to fit around flow conditioners 1110 or to permit flow conditioners 1110 to pass through. The physical dimension of flow conditioners 1110 can further be configured to allow flow conditioners 1110 to collapse and expand with expandable body 1112 (e.g., to fit within a delivery catheter). The physical dimensions of flow conditioners 1110 can further be configured to prevent flow conditioners 1110 from occluding a vessel of chamber of heart H in which device 1100 is implanted. That is, a length and/or width of flow conditioners 1110 can be relatively short enough so that flow conditioners 1110 do not protrude from device 1100 and extend fully across a vessel or chamber of heart H and block blood flow. The physical dimensions of flow conditioners 1110 can further be configured to modify a hemodynamic characteristic (e.g., helicity, vorticity, velocity, turbulence, flow direction, etc.) of blood flowing through or out of device 1100 in a particular manner.

One or more flow conditioners 1110 can be positioned in any suitable arrangement with respect to body 1112 of device 1100. In some examples, flow conditioners 1110 can be arranged about a circumference of inner surface 1132 of central flow tube 1126. In some examples, flow conditioners 1110 can be positioned adjacent central flow tube 1126 but connected to arms 1130. In some examples, flow conditioners 1110 can be located adjacent inflow end 1118 and/or outflow end 1120 of central flow tube 1126. In some examples, flow conditioners 1110 are connected to ones of struts 1122 that form a first row of openings 1124, the first row of openings 1124 being adjacent to inflow end 1118. In some examples, flow conditioners 1110 are connected to ones of struts 1122 that form a second row of openings 1124, the second row of openings 1124 being adjacent to outflow end 1120. Device 1100 can include any number of flow conditioners 1110 in any one or more of the foregoing locations. Locations of flow conditioners 1110 can be configured to prevent flow conditioners 1110 from interfering with other parts of device 1100 or adjacent tissue walls. In other examples, components of device 1100 can be designed to fit around flow conditioners 1110 or to permit flow conditioners 1110 to pass through. The locations of flow conditioners 1110 can further be configured to allow flow conditioners 1110 to collapse and expand with expandable body 1112 (e.g., to fit within a delivery catheter). The locations of flow conditioners 1110 can further be configured to prevent flow conditioners 1110 from occluding a vessel of chamber of heart H in which device 1100 is implanted. The locations of flow conditioners 1110 can further be configured to modify a hemodynamic characteristic (e.g., helicity, vorticity, velocity, turbulence, flow direction, etc.) of blood flowing through or out of device 1100 in a particular manner.

Once device 1100 is implanted in the cardiovascular system (e.g., inter-atrial septum IS or the tissue wall between left atrium LA and coronary sinus CS), circulating blood passes through device 1100. As blood flows into, through, and out of device 1100 along flow axis FL, flow conditioners 1110 interact with the blood flow to modify or affect a hemodynamic characteristic (e.g., helicity, vorticity, velocity, turbulence, flow direction, etc.) of the flow. Flow conditioners 1110 can interact with blood flowing through or out of device 1100 by adding flow resistance and/or changing the direction of the blood flow to prevent reversal of blood flow. For example, flow conditioners 1110 may increase or decrease vorticity or helicity of the flow. In some examples, flow conditioners 1110 may cause the flow to be smoother (decrease the turbulence). In other examples, flow conditioners 1110 can increase turbulence in the flow. In some examples, flow conditioners 1110 can change a flow direction of the flow. In some examples, flow conditioners 1110 can align the flow with a natural vortical flow pattern of blood through a vessel or chamber of heart H, such as the left-sided flow vortex in left atrium LA or the right-sided flow vortex in right atrium RA. In other examples, flow conditioners 1110 can align the flow with a natural helical flow pattern of blood through a vessel or chamber of heart H, such as helical flow in coronary sinus CS. Flow conditioners 1110 that are located circumferentially at inflow end 1118 and/or outflow end 1120 of device 1100 can create helical flow patterns near an adjacent vessel or chamber wall to force blood to flow back towards the center of the vessel or chamber in a same helical direction, thereby producing forward movement of the blood. More generally, flow conditioners 1110 adjacent to inflow end 1118 can modify a hemodynamic characteristic of blood flowing through central flow tube 1126, and flow conditioners 1110 adjacent to outflow end 1120 can modify a hemodynamic characteristic of blood flowing out of central flow tube 1126.

Shunt device 1100, including flow conditioners 1110, can produce hemodynamic effects to minimize disruption to or enhance the natural flow patterns in heart H, such as the left-sided flow vortex in left atrium LA, the right-sided flow vortex in right atrium RA, and/or helical flow in coronary sinus CS. Flow conditioners 1110 can modify hemodynamic characteristics of blood flowing through or out of device 1100 such that (a) any disruptions to the natural flow patterns that would be caused by an implantable device are minimized; (b) reduced flow due to a pathophysiology or other cause is mitigated; and/or (c) baseline flow is enhanced. As a result, shunt device 1100 can maintain kinetic energy of the cardiovascular blood flow, which in turn reduces the cardiac work needed and improves cardiac efficiency. These hemodynamic effects can potentially improve patient outcomes after receiving shunt device 1100 because device 1100 can be more effective and potentially safer. At the same time, flow conditioners 1110 can be incorporated relatively easily into device 1100 in many configurations, so different variations of device 1100 can be optimized for use in many different scenarios (e.g., for many different patient conditions).

FIGS. 11A-11B will be described together. FIG. 11A is an enlarged partial perspective view illustrating fin-type flow conditioner 1140 interacting with low blood flow through shunt device 1100. FIG. 11B is an enlarged partial perspective view illustrating fin-type flow conditioner 1140 interacting with high blood flow through shunt device 1100.

FIGS. 11A-11B show a portion of body 1112 (of shunt device 1100) including struts 1122, fin-type flow conditioner 1140 including attachment region 1142, joint 1144, and bias member 1146. FIGS. 11A-11B also show attachment angle 1148 (FIG. 11A), deflection angle 1149 (FIG. 11B), fin longitudinal axis 1150, and strut longitudinal axis 1152 (of a corresponding one of struts 1122).

Flow conditioner 1140 is one example of fin-type flow conditioners 1110 as described above with reference to FIGS. 9-10. Flow conditioner 1140 has one or more attachment regions 1142 along its length (e.g., from a root portion to a tip portion). Attachment region 1142 is a region where flow conditioner 1140 is connected to body 1112 at one of struts 1122. In some examples, attachment region 1142 can be a single point. Attachment angle 1148 is formed between fin longitudinal axis 1150 of flow conditioner 1140 and strut longitudinal axis 1152 of a corresponding one of struts 1122 to which flow conditioner 1140 is attached. Attachment angle 1148, as illustrated in FIG. 11A, is a position of flow conditioner 1140 in a non-deflected or initial state. Flow conditioner 1140 can be angled radially inward from a circumference of inner surface 1132 of central flow tube 1126 of body 1112 at attachment angle 1148. In some examples, attachment angle 1148 is ninety degrees or less. In other examples, attachment angle 1148 is greater than ninety degrees. For example, attachment angle 1148 can be selected so that flow conditioner 1140 is angled away from other components of device 1100 or so that flow conditioner 1140 interacts optimally with blood flowing through or out of device 1100. Deflection angle 1149, as illustrated in FIG. 11B, is formed between fin longitudinal axis 1150 and strut longitudinal axis 1152 when flow conditioner 1140 is deflected. Deflection angle 1149 represents a change from attachment angle 1148. Generally, deflection angle 1149 is smaller than attachment angle 1148. Attachment angle 1148 and/or deflection angle 1149 can be selected or calibrated to optimize the position of flow conditioner 1140 based on a low heart rate or a high heart rate and low or high velocity blood flow.

Flow conditioner 1140 is attached to body 1112 at attachment region 1142, thereby forming joint 1144 with a corresponding portion of body 1112. More specifically, joint 1144 is formed between flow conditioner 1140 and one of struts 1122. Joint 1144 can include any suitable attachment mechanism, such as a hinge, a flexible section of tissue or another material, a spring, etc. In some examples, joint 1144 is a flexible joint that readily permits deflection of flow conditioner 1140. In other examples, joint 1144 is a rigid joint (e.g., if flow conditioner 1140 is integrally formed with the corresponding strut 1122).

Flow conditioner 1140 can also be biased away from body 1112 by bias member 1146, which can be a spring or other suitable feature for biasing flow conditioner 140. For example, when flow conditioner 1140 is connected to inner surface 1132 of central flow tube 1126, flow conditioner 1140 can be biased radially inward from a circumference of inner surface 1132. Bias member 1146 is attached to one or more of struts 1122. In some examples, joint 1144 and bias member 1146 are on a same one of struts 1122. In other examples, joint 1144 can be on a first one of struts 1122 and bias member 1146 can be attached to a second one of struts 1122. In some such examples, the second one of struts 1122 to which bias member 1146 is attached can be adjacent to the first one of struts 1122. In yet other examples, device 1100 does not include bias member 1146.

As illustrated in FIG. 11A, flow conditioner 1140 is in a first, or non-deflected, position when there is low blood flow or when relatively lower velocity blood flows through device 1100. The non-deflected position is represented by attachment angle 1148. As illustrated in FIG. 11B, flow conditioner 1140 deflects toward body 1112 when there is increased blood flow or when relatively higher velocity blood flows through device 1100. The deflected flow conditioner 1140 is at deflection angle 1149, which can be a smaller angle compared to attachment angle 1148. Additionally, bias member 1146 will be compressed back toward body 1112.

The attachment angle 1148 at joint 1144 allows the initial positioning of flow conditioners 1140 to be adjusted based on a desired hemodynamic effect. Moreover, the amount of disruption (or flow modification) caused by flow conditioner 1140 on blood flowing through or out of device 1100 can be calibrated at attachment angle 1148 or by the incorporation of bias member 1146. Specifically, the amount of deflection of flow conditioner 1140 in response to increased blood flow (or increased velocity of blood flow) can be controlled by a tension of bias member 1146. Bias member 1146 can be adjusted to permit greater or lesser deflection of flow conditioner 1140 based on a desired interaction between flow conditioner 1140 and blood flowing through or out of device 1100. Additionally, deflectable flow conditioners 1140 can have increased compliance. These characteristics allow flow conditioner 1140 to be flexibly implemented in a wide variety of implantable devices to cause different hemodynamic effects, and the positioning of flow conditioner 1140 can be tuned for each implementation.

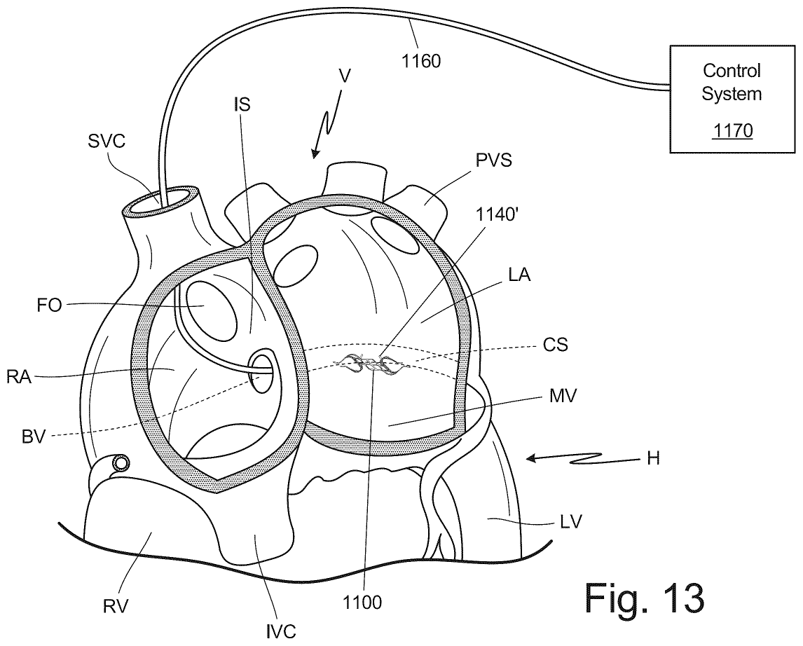

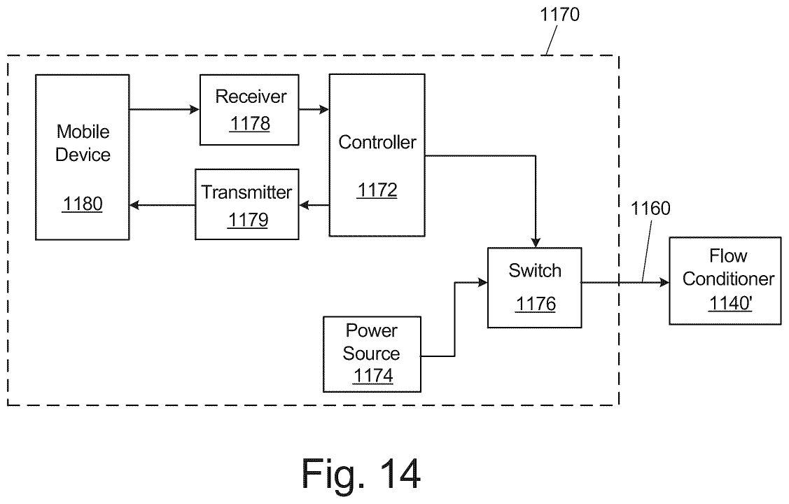

FIGS. 12A-14 will be described together. FIG. 12A is a schematic diagram illustrating connection of control components to actively controlled flow conditioner 1140′. FIG. 12B is a schematic diagram illustrating electromechanical actuation of actively controlled flow conditioner 1140′. FIG. 13 is a sectional view of heart H illustrating an example positioning of the control components for actively controlled flow conditioner 1140′. FIG. 14 is a schematic diagram illustrating control system 1170 for actively controlled flow conditioner 1140′.

FIGS. 12A-12B show a portion of body 1112 (of shunt device 1100) including struts 1122, flow conditioner 1140′ including extension region 1142′, electrical connectors 1160, and control system 1170. FIGS. 12A-12B also show extension angle 1148′, adjusted angle 1149′, fin longitudinal axis 1150, and strut longitudinal axis 1152 (of a corresponding one of struts 1122). FIG. 13 shows shunt device 1100 including actively controlled flow conditioners 1140′, electrical connectors 1160, and control system 1170. FIG. 13 also shows heart H, vasculature V, right atrium RA, right ventricle RV, left atrium LA, left ventricle LV, superior vena cava SVC, inferior vena cava IVC, pulmonary veins PVS, mitral valve MV, coronary sinus CS, thebesian valve BV, inter-atrial septum IS, and fossa ovalis FO. FIG. 14 shows actively controlled flow conditioner 1140′, electrical connectors 1160, and control system 1170, which includes controller 1172, power source 1174, switch 1176, receiver 1178, transmitter 1179, and mobile device 1180.

Flow conditioner 1140′ includes a similar structure and function as described above with respect to flow conditioners 1110 shown in FIGS. 9-10 and flow conditioner 1140 shown in FIGS. 11A-11B, except flow conditioner 1140′ is electromechanically actuated by control system 1170.

Flow conditioner 1140′ can be monolithically formed with one or more of struts 1122 of body 1112. More specifically, flow conditioner 1140′ and a portion of body 1112 to which flow conditioner 1140′ is connected (one or more struts 1122) can be monolithically formed of a shape-memory alloy, such as nitinol. In other examples, flow conditioner 1140′ can be connected to body 1112 by an electrically controllable mechanism, such as a motorized hinge. Flow conditioner 1140′ has one or more extension regions 1142′ along its length (e.g., from a root portion to a tip portion). Extension region 1142′ is a region where flow conditioner 1140′ extends from body 1112 at one (or more) of struts 1122. In some examples, flow conditioner 1140′ extends monolithically from body 1112 at extension region 1142′.

Extension angle 1148′ is formed between fin longitudinal axis 1150 of flow conditioner 1140′ and strut longitudinal axis 1152 of a corresponding one of struts 1122 from which flow conditioner 1140′ extends. Extension angle 1148′, as illustrated in FIG. 12A, is a position of flow conditioner 1140′ in an initial state (e.g., at body temperature). Flow conditioner 1140′ can be angled radially inward from a circumference of inner surface 1132 of central flow tube 1126 (of body 1112) at extension angle 1148′. In some examples, extension angle 1148′ is ninety degrees or less. In other examples, extension angle 1148′ is greater than ninety degrees. For example, extension angle 1148′ can be selected so that flow conditioner 1140′ is angled away from other components of device 1100 or so that flow conditioner 1140 interacts optimally with blood flowing through or out of device 1100. Moreover, the angle of flow conditioner 1140′ is controllable during electromechanical actuation of flow conditioner 1140′. In some examples, the angle of flow conditioner 1140′ is controllable based on preset shapes of the shape-memory alloy that makes up at least the portion of body 1112 from which flow conditioner 1140′ extends. Adjusted angle 1149′ is formed between fin longitudinal axis 1150 and strut longitudinal axis 1152 when flow conditioner 1140′ is electromechanically actuated. That is, in examples where flow conditioner 1140′ and a corresponding portion of body 1112 are monolithically formed, different adjusted angles 1149′ of flow conditioner 1140′ can be preset and remembered by the shape-memory alloy when current is applied to flow conditioner 1140′ to cause the shape-memory alloy to reach its transformation temperature. Adjusted angle 1149′ represents a change from extension angle 1148′. Generally, adjusted angle 1149′ is smaller than extension angle 1148′. Extension angle 1148′ and/or adjusted angle 1149′ can be selected or calibrated to optimize the position of flow conditioner 1140′ based on a low heart rate or a high heart rate and low or high velocity blood flow.

Electrical connectors 1160 are electrical connections between flow conditioner 1140′ and control system 1170. As shown in FIG. 13, electrical connectors 1160 extend from device 1100 through coronary sinus into right atrium RA and through superior vena cava SVC. Electrical connectors 1160 can generally be placed or fed through the right side of the cardiovascular system to avoid larger arteries, such as aorta AT, and to stay within veins, such as superior vena cava SVC and the subclavian vein (not shown). That is, although not shown in FIG. 13, electrical connectors 1160 can extend from superior vena cava SVC through a portion of the thoracic vasculature (e.g., the subclavian vein) and out of the body via a puncture or incision to an externally located control system 1170. In other examples, electrical connectors 1160 can be placed in or along any vessels or chambers of heart H that are convenient with respect to the location of shunt device 1100. As shown in FIG. 14, electrical connectors 1160 are connected to switch 1176 of control system 1170.

Control system 1170 is a system of components for controlling (e.g., electromechanically actuating) flow conditioners 1140′. Control system 1170 can include wired or wireless connections between components. Moreover, all or some of components of control system 1170 can be externally located outside the body. In one example, control system 1170 can include receiver 1178 and transmitter 1179 in order to wirelessly communicate with mobile device 1180 to receive and send signals for controlling flow conditioners 1140′. In some examples, receiver 1178 and transmitter 1179 can be a transceiver. In other examples, control system 1170 may not include receiver 1178, transmitter 1179, and mobile device 1180, and instead controller 1172 can directly implement predefined process instructions for controlling flow conditioners 1140′. Alternatively, control system 1170 may not include controller 1172, and instead switch 1176 can be manually activated to supply current from power source 1174 to flow conditioners 1140′.

Controller 1172 is configured to implement process instructions for operational control of flow conditioners 1140′. For instance, controller 1172 can include one or more processors and computer-readable memory configured to implement functionality and/or process instructions for execution within control system 1170. Examples of one or more processors can include, e.g., any one or more of a microprocessor, a controller, a digital signal processor (DSP), an application specific integrated circuit (ASIC), a field-programmable gate array (FPGA), or other equivalent discrete or integrated logic circuitry.

Computer-readable memory of controller 1172 can be configured to store information used by controller 1172 during operation of control system 1170. Computer-readable memory, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). In some examples, the computer-readable memory is used to store program instructions for execution by the one or more processors. Computer-readable memory, in one example, is used by software or applications running on controller 1172 to temporarily store information during program execution. Computer-readable memory can include volatile and non-volatile memories. Examples of volatile memories can include, e.g., random access memories (RAM), dynamic random access memories (DRAM), static random access memories (SRAM), and other forms of volatile memories. Examples of non-volatile storage elements can include, for example, magnetic hard discs, optical discs, flash memories, or forms of electrically programmable memories (EPROM) or electrically erasable and programmable (EEPROM) memories.

Examples of controller 1172 can include any one or more of microcontrollers or other computers. Controller 1172 can be configured to communicate with any one or more of the components of control system 1170, including switch 1176, receiver 1178, and transmitter 1179. Although the example of FIG. 14 illustrates controller 1172 as operatively coupled to other components of control system 1170, other examples can include a dedicated device where controller 1172 is integrated with switch 1176, receiver 1178, and transmitter 1179 to control flow conditioners 1140′.

Power source 1174 supplies current to flow conditioner 1140′ via electrical connectors 1160. In some examples, power source 1174 can be a battery. Switch 1176 is between power source 1174 and flow conditioner 1140′. Switch 1176 can be activated (or turned on) such that current can flow from power source 1174 to flow conditioner 1140′. Switch 1176 can also be deactivated (or turned off) such that current does not flow from power source 1174 to flow conditioner 1140′. Switch 1176 can be activated or deactivated manually or by a control signal from controller 1172. For example, switch 1176 can be a metal-oxide-semiconductor field-effect transistor (MOSFET) or any other electrical switch.

Controller 1172 is connected to or includes receiver 1178 and transmitter 1179 (or a transceiver rather than separate transmitter and receiver) for sending and receiving wireless signals. For example, receiver 1178 can receive a Bluetooth Low Energy (BLE) signal. In other examples, receiver 1178 can receive a Wi-Fi signal. In yet other examples, receiver 1178 can be a receiver for any suitable wireless signal type. Similarly, transmitter 1179 can be a transmitter for any suitable wireless signal type. In some examples, receiver 1178 can receive signals from mobile device 1180, and transmitter 1179 can transmit signals to mobile device 1180.

Mobile device 1180 is an access point for remotely controlling flow conditioners 1140′. For example, mobile device 1180 can be a cell phone, tablet, or other device capable of sending a wireless signal to receiver 1178 to communicate with controller 1172. Mobile device 1180 can include a user interface (UI) for displaying control options for control system 1170 to a user, such as a physician. Mobile device 1180 can include a display and/or other UI elements (e.g., keyboard, buttons, monitor, graphical control elements presented at a touch-sensitive display, or other UI elements). In some examples, mobile device includes a graphical user interface (GUI) that includes graphical representations of control options for control system 1170, such as graphical representations of a button for activating switch 1176.

In operation of control system 1170, switch 1176 is activated so that current can flow from power source 1174 to flow conditioner 1140′ along electrical connectors 1160. Switch 1176 can be manually activated or can be activated based on a signal from controller 1172. Controller 1172 can send signals based on predefined instructions, such as configurations, stored thereon or can receive a signal from mobile device 1180 via receiver 1178. The supplied current causes flow conditioner 1140′ to deflect to adjusted angle 1149′. In some examples, flow conditioner 1140′ that is formed of a shape-memory alloy reaches its transformation temperature and shifts to a preset shape that coincides with the transformation temperature. That is, as illustrated in FIG. 12B, flow conditioner 1140′ will deflect from extension angle 1148′ to adjusted angle 1149′ based on preset shapes of the shape-memory alloy. In other examples, the supplied current activates an electrically controllable hinge or other active mechanism at extension region 1142′. On the other hand, when switch 1176 is deactivated, current is no longer supplied to flow conditioner 1140′, and flow conditioner 1140′ will return to its initial position at extension angle 1148′, as illustrated in FIG. 12A.

Control system 1170 allows the positioning of flow conditioner 1140′ to be actively controlled or adjusted once device 1100 has been implanted in the cardiovascular system. That is, the amount of disruption (or flow modification) caused by flow conditioners 1140′ on blood flowing through or out of device 1 100 can be actively calibrated by changing extension angle 1148′ to adjusted angle 1149′ in response to supplied current from power source 1174. In this way, a user such as a physician or a patient can adjust the position of flow conditioner 1140′ based on changed conditions without having to directly access flow conditioner 1140′ (e.g., via a surgical procedure or other route). For example, in response to a worsening heart disease, the position of flow conditioner 1140′ could be adjusted to have a more significant effect on hemodynamics. Further, control system 1170 using mobile device 1180 allows remote control and adjustment of the positioning of flow conditioner 1140′, which may streamline a procedure for adjusting the positioning of flow conditioner 1140′ or provide a user-friendly alternative option for adjusting the positioning of flow conditioner 1140′. Forming flow conditioner 1140′ and a corresponding portion of body 1112 of a shape-memory alloy also allows for active control of flow conditioner 1140′ by a relatively minor modification to the structure of device 1100.

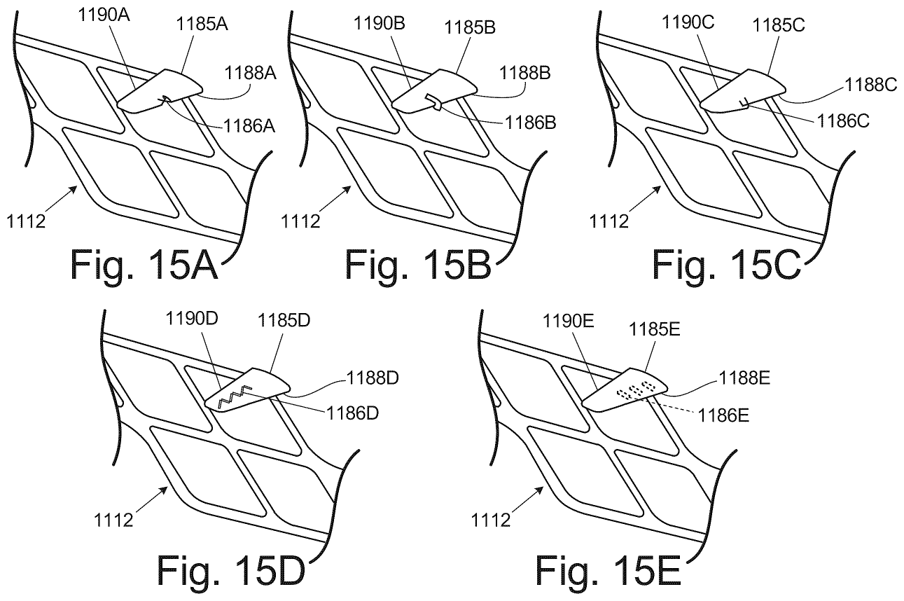

FIGS. 15A-15E will be described together. FIGS. 15A-15E are enlarged partial perspective views of body 1112 of shunt device 1100 illustrating several variations of fin-type flow conditioners. The fin-type flow conditioners described herein can take a number of different forms. Five examples are provided with reference to FIGS. 15A-15E. These examples are not intended to be limiting, and other examples are possible. FIG. 15A shows flow conditioner 1185A, attached to a portion of body 1112 and including flow microfeature 1186A. Flow conditioner 1185A further includes leading edge 1188A and trailing edge 1190A. FIG. 15B shows flow conditioner 1185B, attached to a portion of body 1112 and including flow microfeature 1186B. Flow conditioner 1185B further includes leading edge 1188B and trailing edge 1190B. FIG. 15C shows flow conditioner 1185C, attached to a portion of body 1112 and including flow microfeature 1186C. Flow conditioner 1185C further includes leading edge 1188C and trailing edge 1190C. FIG. 15D shows flow conditioner 1185D, attached to a portion of body 1112 and including flow microfeature 1186D. Flow conditioner 1185D further includes leading edge 1188D and trailing edge 1190D. FIG. 15E shows flow conditioner 1185E, attached to a portion of body 1112 and including flow microfeature 1186E. Flow conditioner 1185E further includes leading edge 1188E and trailing edge 1190E.

Each of flow conditioners 1185A, 1185B, 1185C, 1185D, and 1185E can one of fin-type flow conditioners 1110 shown in FIGS. 9-10, fin-type flow conditioner 1140 shown in FIGS. 11A-11B, or fin-type flow conditioner 1140′ shown in FIGS. 12A-14. Specifically, each of flow conditioners 1185A, 1185B, 1185C, 1185D, and 1185E can be an airfoil. Flow conditioner 1185A extends between leading edge 1188A and trailing edge 1190A, flow conditioner 1185B extends between leading edge 1188B and trailing edge 1190B, flow conditioner 1185C extends between leading edge 1188C and trailing edge 1190C, flow conditioner 1185D extends between leading edge 1188D and trailing edge 1190D, and flow conditioner 1185E extends between leading edge 1188E and trailing edge 1190E.

Flow conditioner 1185A includes flow microfeature 1186A, which is a leading edge notch. Flow conditioner 1185B includes flow microfeature 1186B, which is a boundary layer fence. Flow conditioner 1185C includes flow microfeature 1186C, which is a leading edge dogtooth. Flow conditioner 1185D includes flow microfeature 1186D, which is a group of vortex generators. Flow conditioner 1185E includes flow microfeature 1186E, which is a group of vortilons (shown in FIG. 15E with illusion lines through flow conditioner 1185E from an opposite side of flow conditioner 1185E with respect to the viewing plane). For example, flow microfeatures 1186A, 1186B, 1186C, 1186D, and 1186E can be located at or proximal to the respective leading edge 1188A, 1188B, 1188C, 1188D, and 1188E of the corresponding flow conditioner 1185A, 1185B, 1185C, 1185D, and 1185E. As shown in FIGS. 15A-15C, flow microfeatures 1186A, 1186B, and 1186C are located along respective leading edges 1188A, 1188B, and 1188C. As shown in FIGS. 15D-15E, flow microfeatures 1186D and 1186E are located proximal to respective leading edges 1188D and 1188E. Other fin-type flow conditioners can include a combination of the flow microfeatures described herein. Further, device 1100 can include any combination of flow conditioners 1185A, 1185B, 1185C, 1185D, and 1185E and flow microfeatures 1186A, 1186B, 1186C, 1186D, and 1186E in any suitable pattern or organization, depending on the desired hemodynamic characteristics.

Flow microfeatures 1186A, 1186B, 1186C, 1186D, and 1186E interact with blood flowing through or out of device 1100 as it reaches flow conditioners 1185A, 1185B, 1185C, 1185D, and 1185E. Each of flow microfeatures 1186A, 1186B, 1186C, 1186D, and 1186E can produce characteristic flow effects on the flow of blood through or out of device 1100.

Flow conditioners 1185A, 1185B, 1185C, 1185D, and 1185E including flow microfeatures 1186A, 1186B, 1186C, 1186D, and 1186E provide a wider range of options and greater flexibility for designing shunt devices to modify hemodynamic characteristics for producing desired hemodynamic effects on blood flowing through or out of the device. Incorporating flow microfeatures 1186A, 1186B, 1186C, 1186D, and 1186E or combinations of flow microfeatures 1186A, 1186B, 1186C, 1186D, and 1186E can result in finer or more granular control of hemodynamic effects.

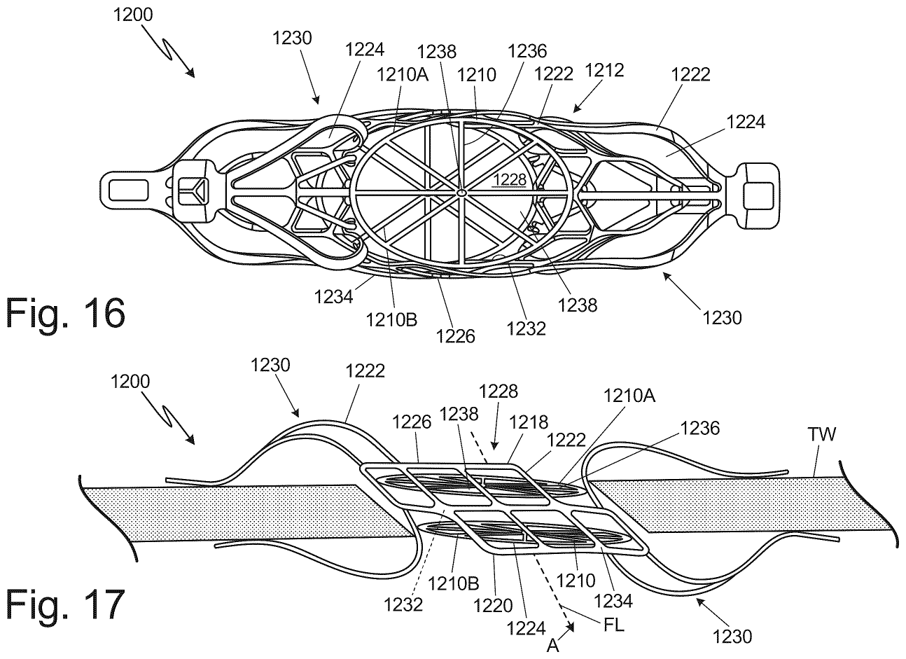

FIGS. 16-17 will be described together. FIG. 16 is a bottom view of shunt device 1200 including plate-type flow conditioners 1210. FIG. 17 is a side view of shunt device 1200 anchored to tissue wall TW and including plate-type flow conditioners 1210. FIGS. 16-17 show shunt device 1200 including plate-type flow conditioners 1210, including flow conditioner 1210A and 1210B (which will be referred to collectively herein by the shared reference number), body 1212, inflow end 1218, and outflow end 1220. Body 1212 is formed of struts 1222 and openings 1224. Body 1212 includes central flow tube 1226, flow path 1228, and arms 1230, and defines flow axis FL. Central flow tube 1226 includes inner surface 1232 and outer surface 1234. Flow conditioners 1210 include walls 1236 and define flow passages 1238 therein. FIG. 17 also shows tissue wall TW.

Shunt device 1200 includes generally the same structure and function as shunt device 1100 described above in reference to FIGS. 9-15E, except shunt device 1200 includes plate-type flow conditioners 1210 instead of fin-type flow conditioners (e.g., flow conditioners 1100).

Flow conditioners 1210 are flow plates or plate-type flow conditioners. Flow conditioner 1210A is positioned adjacent to inflow end 1218, and flow conditioner 1210B is positioned adjacent to outflow end 1220. Each individual one of flow conditioners 1210 can also be referred to as a flow conditioner feature. Flow conditioners 1210 can be flattened or cylindrical projections from body 1212. More specifically, flow conditioners 1210 are connected to body 1212 at corresponding ones of struts 1222. Flow conditioners 1210 can be connected to body 1212 at central flow tube 1226 and/or at ones of arms 1230. In some examples, flow conditioners 1210 are attached to multiple struts 1222 along portions of body 1212. In some examples, flow conditioners 1210 are connected circumferentially at several locations along inner surface 1232 of central flow tube 1226. In some examples, flow conditioners 1210 are continuously formed with inner surface 1232 of central flow tube 1226. Flow conditioners 1210 are attached by an attachment mechanism or monolithically formed with a portion of body 1212. Each plate-type flow conditioner 1210 can be a single part rather than a plurality of individual fin-type flow conditioners.

In general, flow conditioners 1210 can take a number of different forms (i.e., shapes, sizes, etc.). Physical dimensions (e.g., length, width, shape, cross-sectional shape, etc.) of flow conditioners 1210 can be configured to prevent flow conditioners 1210 from interfering with other parts of device 1200 or adjacent tissue walls (e.g., tissue wall TW). In other examples, components of device 1200 can be designed to fit around flow conditioners 1210 or to permit flow conditioners 1210 to pass through. The physical dimension of flow conditioners 1210 can further be configured to allow flow conditioners 1210 to collapse and expand with expandable body 1212 (e.g., to fit within a delivery catheter). The physical dimensions of flow conditioners 1210 can further be configured to prevent flow conditioners 1210 from occluding a vessel of chamber of heart H in which device 1200 is implanted. That is, a length and/or width of flow conditioners 1210 can be relatively short enough so that flow conditioners 1210 do not protrude from device 1200 and extend fully across a vessel or chamber of heart H and block blood flow. The physical dimensions of flow conditioners 1210 can further be configured to modify a hemodynamic characteristic (e.g., helicity, vorticity, velocity, turbulence, flow direction, etc.) of blood flowing through or out of device 1200 in a particular manner.

One or more flow conditioners 1210 can be positioned in any suitable arrangement with respect to body 1212 of device 1200. In the example shown in FIGS. 16-17, device 1200 includes two flow conditioners: flow conditioner 1210A and flow conditioner 1210B. Other examples can include any number of flow conditioners 1210. Flow conditioners 1210 are positioned to span across a portion of central flow tube 1226 and flow path 1228 such that flow conditioners 1210 intersect flow axis FL through central flow tube 1226. In some examples, flow conditioners 1210 can be located adjacent to inflow end 1218 (e.g., flow conditioner 1210A) and/or outflow end 1220 (e.g., flow conditioner 210B). In some examples, flow conditioners 1210 can be positioned adjacent central flow tube 1226 but connected to arms 1230. In some examples, flow conditioners 1210 are connected to ones of struts 1222 that form a first row of openings 1224 that is adjacent to inflow end 1218. In some examples, flow conditioners 1210 are connected to ones of struts 1222 that form a second row of openings 1224 that is adjacent to outflow end 1220. Device 1200 can include any number of flow conditioners 1210 in any one or more of the foregoing locations. Locations of flow conditioners 1210 can be configured to prevent flow conditioners 1210 from interfering with other parts of device 1200 or adjacent tissue walls. In other examples, components of device 1200 can be designed to fit around flow conditioners 1210 or to permit flow conditioners 1210 to pass through. The locations of flow conditioners 1210 can further be configured to allow flow conditioners 1210 to collapse and expand with expandable body 1212 (e.g., to fit within a delivery catheter). The locations of flow conditioners 1210 can further be configured to prevent flow conditioners 1210 from occluding a vessel or chamber of heart H in which device 1200 is implanted. The locations of flow conditioners 1210 can further be configured to modify a hemodynamic characteristic (e.g., helicity, vorticity, velocity, turbulence, flow direction, etc.) of blood flowing through or out of device 1200 in a particular manner.

Each flow conditioner 1210 includes walls 1236 that define flow passages 1238 therein. Flow passages 1238 extend through a corresponding one of flow conditioners 1210 such that blood flowing along flow axis FL through central flow tube 1226 within device 1200 can pass through the corresponding flow conditioner 1210. That is, flow passages 1238 extend from an upstream side to a downstream side of the corresponding flow conditioner 1210. The form of each flow conditioner 1210 can depend largely on the configuration of flow passages 1238. Flow passages are bounded, at least partially, by walls 1236. In some examples, flow passages 1238 are closed channels that are surrounded by walls 1236 (as shown in FIGS. 16-17). In other examples, flow passages 1238 can be continuous with adjacent flow passages 1238 by gaps or spaces between walls 1236. Individual walls 1236 can have any height (as measured with respect to flow axis FL). In some examples, walls 1236 can have a same height to form a flat upstream and/or downstream surface of flow conditioner 1210. In other examples, walls 1236 can protrude such that the upstream and/or downstream surface of flow conditioner 1210 is not flat. Flow passages 1238 can take a number of different forms (i.e., shapes, sizes, curvatures, etc.). The example illustrated in FIGS. 16-17 includes eight wedge shaped flow passages 1238 and one central circular flow passage 1238 (when viewed perpendicular to an upstream or downstream side of flow conditioner 1210). Other examples can include any combination of shapes and sizes of flow passages 1238 throughout flow conditioners 1210.

Once device 1200 is implanted in the cardiovascular system (e.g., inter-atrial septum IS or the tissue wall between left atrium LA and coronary sinus CS), circulating blood passes through device 1200. As blood flows into, through, and out of device 1200 along flow axis FL, the blood flows through flow passages 1238 of flow conditioners 1210. Flow conditioners 1210 interact with the blood flow to modify or affect a hemodynamic characteristic (e.g., helicity, vorticity, velocity, turbulence, flow direction, etc.) of the flow. Flow conditioners 1210 can interact with blood flowing through or out of device 1200 by adding flow resistance and/or changing the direction of the blood flow to prevent reversal of blood flow. For example, flow conditioners 1210 may increase or decrease vorticity or helicity of the flow. In some examples, flow conditioners 1210 may cause the flow to be smoother (decrease the turbulence). In other examples, flow conditioners 1210 can increase turbulence in the flow. In some examples, flow conditioners 1210 can change a flow direction of the flow. In some examples, flow conditioners 1210 can align the flow with a natural vortical flow pattern of blood through a vessel or chamber of heart H, such as the left-sided flow vortex in left atrium LA or the right-sided flow vortex in right atrium RA. In other examples, flow conditioners 1210 can align the flow with a natural helical flow pattern of blood though a vessel or chamber of heart H, such as helical flow in coronary sinus CS. Flow conditioners 1210 that are located at inflow end 1218 and/or outflow end 1220 of device 1200 can create helical flow patterns near an adjacent vessel or chamber wall to force blood to flow back towards the center of the vessel or chamber in a same helical direction, thereby producing forward movement of the blood. More generally, flow conditioners 1210 adjacent to inflow end 1218 can modify a hemodynamic characteristic of blood flowing through central flow tube 1226, and flow conditioners 1210 adjacent to outflow end 1220 can modify a hemodynamic characteristic of blood flowing out of central flow tube 1226.

Like device 1100 described above, shunt device 1200, including flow conditioners 1210, can produce hemodynamic effects to minimize disruption to or enhance the natural flow patterns in heart H, such as the left-sided flow vortex in left atrium LA, the right-sided flow vortex in right atrium RA, and/or helical flow in coronary sinus CS. Flow conditioners 1210 can modify hemodynamic characteristics of blood flowing through or out of device 1200 such that (a) any disruptions to the natural flow patterns that would be caused by an implantable device are minimized; (b) reduced flow due to a pathophysiology or other cause is mitigated; and/or (c) baseline flow is enhanced. As a result, shunt device 1200 can maintain kinetic energy of the cardiovascular blood flow, which in turn reduces the cardiac work needed and improves cardiac efficiency. These hemodynamic effects can potentially improve patient outcomes after receiving shunt device 1200 because device 1200 can be more effective and potentially safer. At the same time, flow conditioners 1210 can be incorporated relatively easily into device 1200 in many configurations, so different variations of device 1200 can be optimized for use in many different scenarios (e.g., for many different patient conditions).

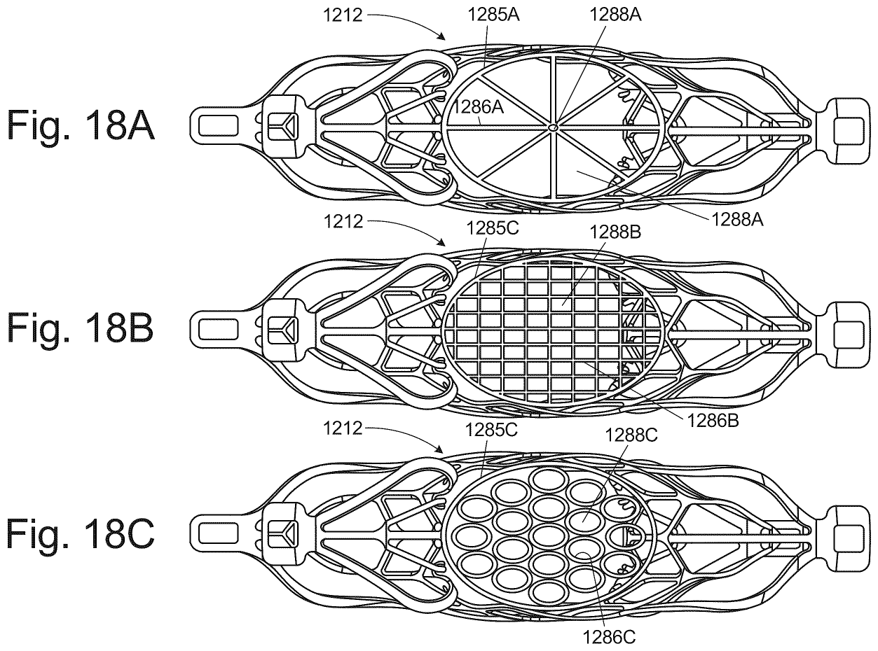

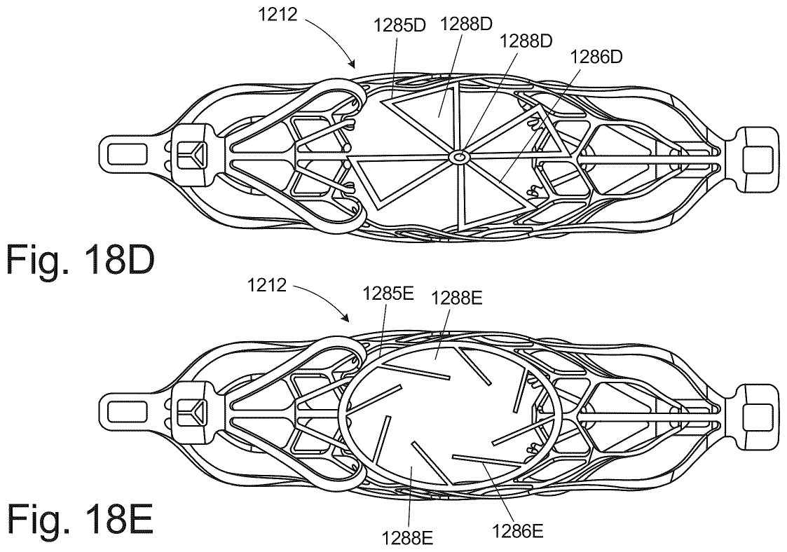

FIGS. 18A-18E will be described together. FIGS. 18A-18E are bottom views of shunt device 1200 illustrating several variations of plate-type flow conditioners. The plate-type flow conditioners described herein can take a number of different forms. Five examples are provided with reference to FIGS. 18A-18E. These examples are not intended to be limiting, and other examples are possible. FIG. 18A shows flow conditioner 1285A attached to body 1212 and including walls 1286A, which define flow passages 1288A therein. FIG. 18B shows flow conditioner 1285B attached to body 1212 and including walls 1286B, which define flow passages 1288B therein. FIG. 18C shows flow conditioner 1285C attached to body 1212 and including walls 1286C, which define flow passages 1288C therein. FIG. 18D shows flow conditioner 1285D attached to body 1212 and including walls 1286D, which define flow passages 1288D therein. FIG. 18E shows flow conditioner 1285E attached to body 1212 and including walls 1286E, which define flow passages 1288E therein.

Each of flow conditioners 1285A, 1285B, 1285C, 1285D, and 1285E can be plate-type flow conditioners 1210 shown in FIGS. 16-17. Flow conditioner 1285A includes walls 1286A, which take the form of connected fins that extend across flow conditioner 1285A in a wheel shape. Accordingly, walls 1286A form flow passages 1288A that have wedge shaped cross sections. Walls 1286A also form a central flow passage 1288A that has a circular cross section. Flow conditioner 1285A is the example of flow conditioners 1210 that is depicted in FIGS. 16-17. Flow conditioner 1285B includes walls 1286B, which take the form of a grid or lattice of flow passages 1288B (such as a Zanker-type flow plate). Flow conditioner 1285C includes walls 1286C, which take the form of connected tubes surrounding flow passages 1288C. Flow conditioner 1285D includes walls 1286D, which take the form of folded vanes in a wheel shape similar to flow conditioner 1285A, except some flow passages 1288D formed by walls 1286D are bounded by an additional wall 1286D rather than body 1212 (e.g., inner surface 1232 of central flow tube 1226 as shown in FIGS. 16-17). Accordingly, walls 1286D form flow passages 1288D that have wedge shaped cross sections. Walls 1286D also form a central flow passage 1288D that has a circular cross section. Flow conditioner 1285E includes walls 1286E, which take the form of angled tabs that form flow passages 1288E therebetween. Flow passages 1288E are continuous with each other centrally. Other plate-type flow conditioners can include a combination of the walls and flow passages described herein. Further, device 1200 can include any combination of flow conditioners 1285A, 1285B, 1285C, 1285D, and 1285E in any suitable pattern or organization, depending on the desired hemodynamic characteristics.

Flow conditioners 1285A, 1285B, 1285C, 1285D, and 1285E interact with blood flowing through or out of device 1200 as it passes through the corresponding flow passages 1288A, 1288B, 1288C, 1288D, and 1288E. Flow conditioners 1285A, 1285B, 1285C, 1285D, and 1285E, each having different respective types or arrangements of walls 1286A, 1286B, 1286C, 1286D, and 1286E and flow passages 1288A, 1288B, 1288C, 1288D, and 1288E, can produce characteristic flow effects on the flow of blood through or out of device 1200.

Flow conditioners 1285A, 1285B, 1285C, 1285D, and 1285E including walls 1286A, 1286B, 1286C, 1286D, and 1286E and flow passages 1288A, 1288B, 1288C, 1288D, and 1288E provide a wider range of options and greater flexibility for designing shunt devices to modify hemodynamic characteristics for producing desired hemodynamic effects on blood flowing through or out of the device. Incorporating flow conditioners 1285A, 1285B, 1285C, 1285D, and 1285E including walls 1286A, 1286B, 1286C, 1286D, and 1286E and flow passages 1288A, 1288B, 1288C, 1288D, and 1288E or combinations of these can result in finer or more granular control of hemodynamic effects.

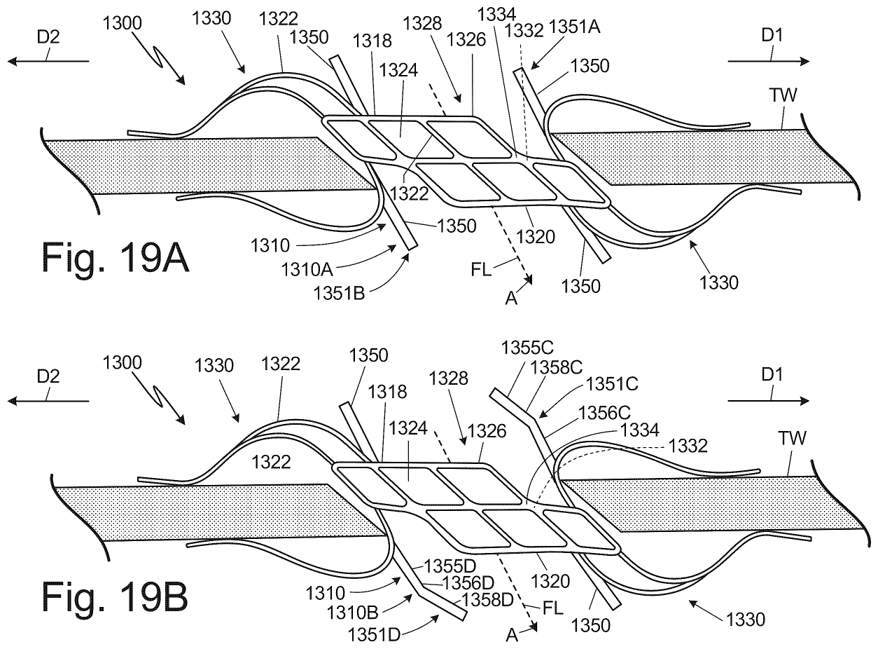

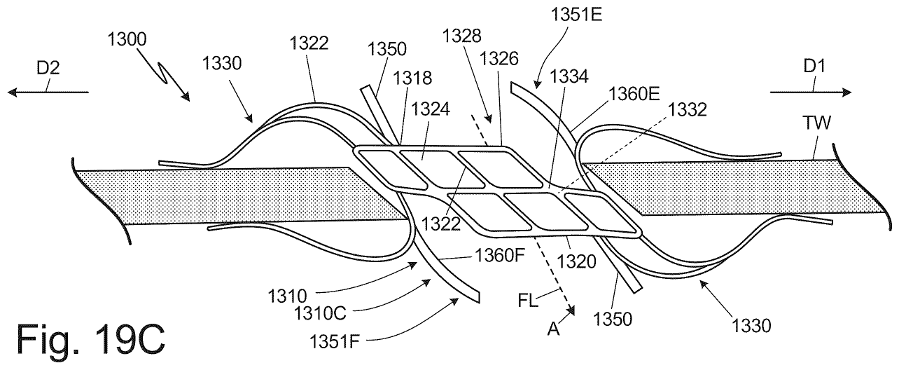

FIGS. 19A-19C will be described together. FIGS. 19A-19C are side views of shunt device 1300 anchored to tissue wall TW and illustrating several variations of deflector-type flow conditioners 1310 for directing fluid flow in one direction. FIG. 19A shows deflector-type flow conditioner 1310A. FIG. 19B shows deflector-type flow conditioner 1310B. FIG. 19C shows deflector-type flow conditioner 1310C. The three non-limiting examples of single direction deflector-type flow conditioners shown in FIGS. 19A-19C (flow conditioners 1310A-1310C) will be referred to collectively herein by the shared reference number 1310.

Shunt device 1300 includes generally the same structure and function as shunt device 1100 described above in reference to FIGS. 9-15E, except shunt device 1300 includes single direction deflector-type flow conditioners 1310 instead of fin-type flow conditioners (e.g., flow conditioners 1110).

FIG. 19A shows shunt device 1300 including single direction deflector-type flow conditioner 1310A, body 1312, inflow end 1318, and outflow end 1320. Body 1312 is formed of struts 1322 and openings 1324. Body 1312 includes central flow tube 1326, flow path 1328, and arms 1330, and defines flow axis FL. Central flow tube 1326 includes inner surface 1332 and outer surface 1334. Flow conditioner 1310A includes deflectors 1350, which are arranged in first set 1351A and second set 1351B. FIG. 19A also shows tissue wall TW, arrow D1 indicating a first direction with respect to tissue wall TW, and arrow D2 indicating a second direction with respect to tissue wall TW.

In the example shown in FIG. 19A, flow conditioner 1310A includes four deflectors 1350. Deflectors 1350 are angled walls or shell-like walls or scoops for directing the flow of blood into or out of device 1300. FIG. 19A shows four deflectors 1350; however, other examples can include more or fewer deflectors 1350. First set 1351A includes two deflectors 1350 connected to body 1312 adjacent inflow end 1318. Second set 1351B includes two deflectors 1350 connected to body 1312 adjacent to outflow end 1320. Although device 1300 is shown in FIG. 19A as including first set 1351A adjacent inflow end 1318 and second set 1351B adjacent outflow end 1320, other examples can include either first set 1351A or second set 1351B instead of both sets. Deflectors 1350 extend parallel to flow axis FL. Using tissue wall TW as a reference plane, deflectors 1350 in first set 1351A are angled in the second direction with respect to tissue wall TW that is denoted by arrow D2, and deflectors 1350 in second set 1351B are angled in the first direction with respect to tissue wall TW that is denoted by arrow D1.

Deflectors 1350 can be connected to central flow tube 1326 and/or arms 1330. In some examples, deflectors 1350 are connected along an arc of the circumference of central flow tube 1326. In some examples, deflectors 1350 span across a portion of central flow tube 1326. In some examples, deflectors 1350 can be positioned adjacent central flow tube 1326 but connected to arms 1330. Locations of deflectors 1350 can be configured to prevent flow conditioner 1310A or individual deflectors 1350 from interfering with other parts of device 1300 or adjacent tissue walls (e.g., tissue wall TW). In other examples, components of device 1300 can be designed to fit around deflectors 1350 or to permit deflectors 1350 to pass through. The locations of deflectors 1350 can further be configured to allow deflectors 1350 to collapse and expand with expandable body 1312 (e.g., to fit within a delivery catheter). The locations of deflectors 1350 can further be configured to prevent deflectors 1350 from occluding a vessel or chamber of heart H in which device 1300 is implanted. The locations of deflectors 1350 can further be configured to modify a hemodynamic characteristic (e.g., helicity, vorticity, velocity, turbulence, flow direction, etc.) of blood flowing into, through, or out of device 1300 in a particular manner.

In general, deflectors 1350 can take a number of different forms (i.e., shapes, sizes, etc.). Physical dimensions (e.g., length, width, shape, cross-sectional shape, etc.) of deflectors 1350 can be configured to prevent flow conditioner 1310A from interfering with other parts of device 1300 or adjacent tissue walls (e.g., tissue wall TW). In other examples, components of device 1300 can be designed to fit around deflectors 1350 or to permit deflectors 1350 to pass through. The physical dimension of deflectors 1350 can further be configured to allow deflectors 1350 to collapse and expand with expandable body 1312 (e.g., to fit within a delivery catheter). The physical dimensions of deflectors 1350 can further be configured to prevent deflectors 1350 from occluding a vessel of chamber of heart H in which device 1300 is implanted. That is, a length and/or width of deflectors 1350 can be relatively short enough so that deflectors 1350 do not protrude from device 1300 and extend fully across a vessel or chamber of heart H and block blood flow. The physical dimensions of deflectors 1350 can further be configured to modify a hemodynamic characteristic (e.g., helicity, vorticity, velocity, turbulence, flow direction, etc.) of blood flowing into, through, or out of device 1300 in a particular manner.

FIG. 19B shows shunt device 1300 including single direction deflector-type flow conditioner 1310B, body 1312, inflow end 1318, and outflow end 1320. Body 1312 is formed of struts 1322 and openings 1324. Body 1312 includes central flow tube 1326, flow path 1328, and arms 1330, and defines flow axis FL. Central flow tube 1326 includes inner surface 1332 and outer surface 1334. Flow conditioner 1310B is arranged in first set 1351C and second set 1351D and includes deflector 1350, primary deflector 1355C, and primary deflector 1355D. Primary deflector 1355C includes first portion 1356C and second portion 1358C. Primary deflector 1355D includes first portion 1356D and second portion 1358D. FIG. 19B also shows tissue wall TW, arrow D1 indicating a first direction with respect to tissue wall TW, and arrow D2 indicating a second direction with respect to tissue wall TW

In the example shown in FIG. 19B, flow conditioner 1310B includes deflectors 1350, primary deflector 1355C, and primary deflector 1355D. Deflectors 1350 have generally the same design and function as deflectors 1350 shown in FIG. 19A. First set 1351C includes primary deflector 1355C and one deflector 1350 connected to body 1312 adjacent inflow end 1318. Second set 1351D includes primary deflector 1355D and one deflector 1350 connected to body 1312 adjacent outflow end 1320. Although device 1300 is shown in FIG. 19B as including first set 1351C adjacent inflow end 1318 and second set 1351D adjacent outflow end 1320, other examples can include either first set 1351C or second set 1351D instead of both sets.

At inflow end 1318, primary deflector 1355C is positioned on a side of device 1300 that extends in the first direction with respect to tissue wall TW that is denoted by arrow D1. One or more deflectors 1350 are positioned on an opposite side of device 1300 from primary deflector 1355C. In some examples, primary deflector 1355C is longer than deflector 1350. Using tissue wall TW as a reference plane, primary deflector 1355C is angled in the second direction with respect to tissue wall TW that is denoted by arrow D2. Primary deflector 1355C includes first portion 1356C connected to body 1312. First portion 1356C extends parallel to flow axis FL. Primary deflector 1355C also includes second portion 1358C connected to first portion 1356C. Second portion 1358C is angled toward flow axis FL in the direction of arrow D2. In other words, a longitudinal axis drawn through second portion 1358C would intersect flow axis FL. First portion 1356C and second portion 1358C can have any suitable respective lengths.

At outflow end 1320, primary deflector 1355D is positioned on a side of device 1300 that extends in the second direction with respect to tissue wall TW that is denoted by arrow D2. One or more deflectors 1350 are positioned on an opposite side of device 1300 from primary deflector 1355D. In some examples, primary deflector 1355D is longer than deflector 1350. Using tissue wall TW as a reference plane, primary deflector 1355D is angled in the first direction with respect to tissue wall TW that is denoted by arrow D1. Primary deflector 1355D includes first portion 1356D connected to body 1312. First portion 1356D extends parallel to flow axis FL. Primary deflector 1355D also includes second portion 1358D connected to first portion 1356D. Second portion 1358D is angled toward flow axis FL in the direction of arrow D1. In other words, a longitudinal axis drawn through second portion 1358D would intersect flow axis FL. First portion 1356D and second portion 1358D can have any suitable respective lengths.

FIG. 19C shows shunt device 1300 including single direction deflector-type flow conditioner 1310C, body 1312, inflow end 1318, and outflow end 1320. Body 1312 is formed of struts 1322 and openings 1324. Body 1312 includes central flow tube 1326, flow path 1328, and arms 1330, and defines flow axis FL. Central flow tube 1326 includes inner surface 1332 and outer surface 1334. Flow conditioner 1310C is arranged in first set 1351E and second set 1351F and includes deflector 1350, curved primary deflector 1360E, and curved primary deflector 1360F. FIG. 19C also shows tissue wall TW, arrow D1 indicating a first direction with respect to tissue wall TW, and arrow D2 indicating a second direction with respect to tissue wall TW

In the example shown in FIG. 19C, flow conditioner 1310C includes deflectors 1350, curved primary deflector 1360E, and curved primary deflector 1360F. Deflectors 1350 have generally the same design and function as deflectors 1350 shown in FIGS. 19A and 19B. First set 1351E includes curved primary deflector 1360E and one deflector 1350 connected to body 1312 adjacent inflow end 1318. Second set 1351F includes curved primary deflector 1360F and one deflector 1350 connected to body 1312 adjacent outflow end 1320. Although device 1300 is shown in FIG. 19C as including first set 1351E adjacent inflow end 1318 and second set 1351F adjacent outflow end 1320, other examples can include either first set 1351E or second set 1351F instead of both sets. Curved primary deflector 1360E is similar to primary deflector 1355C shown in FIG. 19B, except curved primary deflector 1360E is curved toward flow axis FL in the direction of arrow D2. Curved primary deflector 1360F is similar to primary deflector 1355D shown in FIG. 19B, except curved primary deflector 1360F is curved toward flow axis FL in the direction of arrow D1. Curved primary deflectors 1360E and 1360F can have smooth or continuously curved profiles to direct blood flow into or out of device 1300.

Referring to all of FIGS. 19A-19C, once devices 1300 are implanted in the cardiovascular system (e.g., the inter-atrial septum or the tissue wall between the left atrium and the coronary sinus), circulating blood passes through devices 1300. As blood flows into, through, and out of devices 1300 along flow axis FL, flow conditioners 1310 (including deflectors 1350 or a combination of deflectors 1350 and primary deflector 1355C, primary deflector 1355D, curved primary deflector 1360E, or curved primary deflector 1360F) interact with the blood flow to modify or affect a hemodynamic characteristic (e.g., helicity, vorticity, velocity, turbulence, flow direction, etc.) of the flow. First sets 1351A, 1351C, and 1351E of flow conditioners 1310 interact with blood flowing into devices 1300 to direct blood flowing in one direction in a vessel or chamber of heart H into devices 1300 along flow axis FL. Second sets 1351B, 1351D, and 1351F of flow conditioners 1310 change the direction of the blood flow out of devices 1300 to direct the blood flow in one direction. For example, flow conditioners 1310 can direct the blood flow out of devices 1300 to join with a natural blood flow direction and/or pattern in a vessel or chamber of heart H. In some examples, flow conditioners 1310 can direct the flood flow along a tissue wall (e.g., tissue wall TW). In some examples, flow conditioners 1310 can align the flow with a natural vortical flow pattern of blood through a vessel or chamber of heart H, such as the left-sided flow vortex in left atrium LA or the right-sided flow vortex in right atrium RA. In other examples, flow conditioners 1310 can align the flow with a natural helical flow pattern of blood through a vessel or chamber of heart H, such as helical flow in coronary sinus CS.

In addition to the benefits described above with respect to devices 1100 and 1200, incorporating single direction deflector-type flow conditioners 1310 on devices 1300 allow for more precise tuning of flow alignment for blood flowing into and/or out of devices 1300. This can minimize disruption caused by the artificial flow path through shunt devices 1300 branching from a first natural flow path or pattern in a vessel or chamber of heart H and joining with a second natural flow path or pattern in a different vessel or chamber of heart H. Single direction deflector-type flow conditioners 1310 including first sets 1351A, 1351C, and 1351E at inflow end 1318 can mitigate venturi effects that may otherwise cause disruptions where blood flows into devices 1300. Moreover, the natural flow path in a vessel may be in one direction, and single direction deflector-type flow conditioners 1310 including second sets 1351B, 1351D, and 1351F at outflow end 1320 can more effectively align the blood flow out of devices 1300 with that direction.

The single direction deflector-type flow conditioners 1310 described herein can take a number of different forms. Three examples are provided with reference to FIGS. 19A-19C. These examples are not intended to be limiting, and other examples are possible. Yet other examples can include combinations of the features shown in FIGS. 19A-19C.

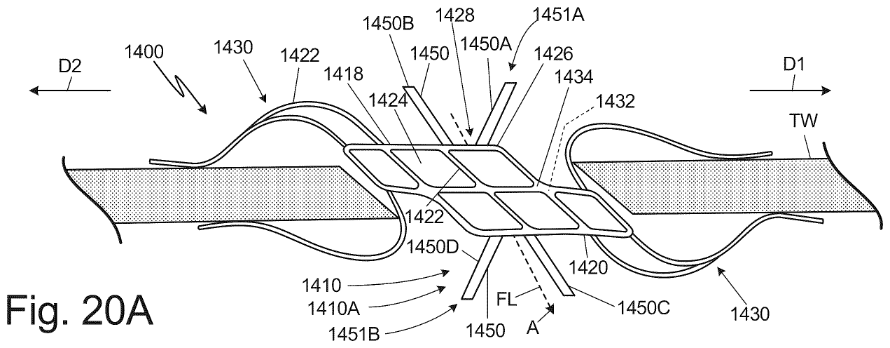

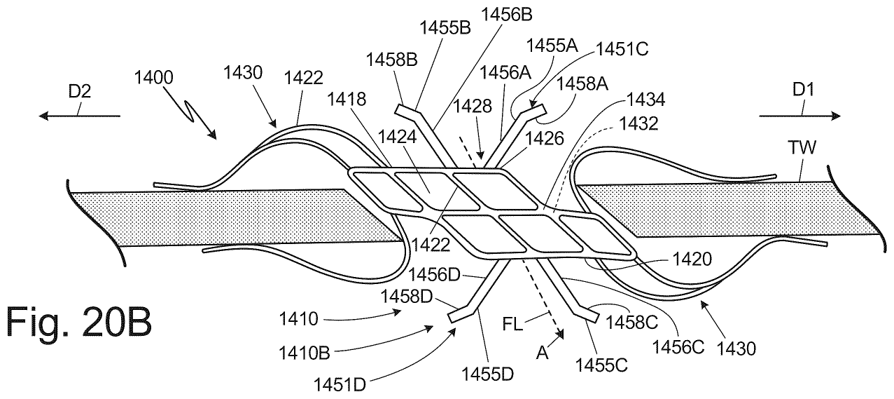

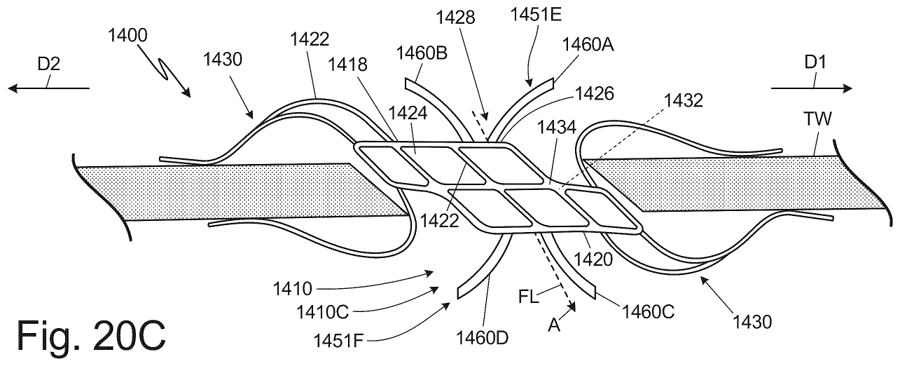

FIGS. 20A-20C will be described together. FIGS. 20A-20C are side views of shunt

device 1400 anchored to tissue wall TW and illustrating several variations of deflector-type flow conditioners 1410 for directing fluid flow in multiple directions. FIG. 20A shows deflector-type flow conditioner 1410A. FIG. 20B shows deflector-type flow conditioner 1410B. FIG. 20C shows deflector-type flow conditioner 1410C. The three non-limiting examples of multi-direction deflector-type flow conditioners shown in FIGS. 20A-20C (flow conditioners 1410A-1410C) will be referred to collectively herein by the shared reference number 1410.

Shunt device 1400 includes generally the same structure and function as shunt device 1100 described above in reference to FIGS. 9-15E, except shunt device 1400 includes multi-direction deflector-type flow conditioners 1410 instead of fin-type flow conditioners (e.g., flow conditioners 1110).

FIG. 20A shows shunt device 1400 including multi-direction deflector-type flow conditioner 1410A, body 1412, inflow end 1418, and outflow end 1420. Body 1412 is formed of struts 1422 and openings 1424. Body 1412 includes central flow tube 1426, flow path 1428, and arms 1430, and defines flow axis FL. Central flow tube 1426 includes inner surface 1432 and outer surface 1434. Flow conditioner 1410A is arranged in first set 1451A and second set 1451B and includes first direction deflector 1450A, second direction deflector 1450B, first direction deflector 1450C, and second direction deflector 1450D (which will be referred to collectively herein by the shared reference number 1450). FIG. 20A also shows tissue wall TW, arrow D1 indicating a first direction with respect to tissue wall TW, and arrow D2 indicating a second direction with respect to tissue wall TW.

Flow conditioner 1410A includes first direction deflector 1450A, second direction deflector 1450B, first direction deflector 1450C, and second direction deflector 1450D. Deflectors 1450 are angled walls or shell-like walls or scoops for directing the flow of blood into or out of device 1400. FIG. 20A shows four deflectors 1450; however, other examples can include more or fewer deflectors 1450. First set 1451A includes two deflectors 1450 (first direction deflector 1450A and second direction deflector 1450B) connected to body 1412 adjacent inflow end 1418. Second set 1451B includes two deflectors 1450 (first direction deflector 1450C and second direction deflector 1450D) connected to body 1412 adjacent to outflow end 1420. Although device 1400 is shown in FIG. 20A as including first set 1451A adjacent inflow end 1418 and second set 1451B adjacent outflow end 1420, other examples can include either first set 1451A or second set 1451B instead of both sets. Deflectors 1450 are angled radially outwards from flow axis FL. Using tissue wall TW as a reference plane, first direction deflectors 1450A and 1450C are angled in the first direction with respect to tissue wall TW that is denoted by arrow D1, and second direction deflectors 1450B and 1450D is angled in the second direction with respect to tissue wall TW that is denoted by arrow D2. Each of deflectors 1450 can have a same angle with respect to flow axis FL or can have different angles.

Deflectors 1450 can extend or span across a portion of central flow tube 1426. Locations of deflectors 1450 can be configured to prevent flow conditioner 1410A or individual deflectors 1450 from interfering with other parts of device 1400 or adjacent tissue walls (e.g., tissue wall TW). In other examples, components of device 1400 can be designed to fit around deflectors 1450 or to permit deflectors 1450 to pass through. The locations of deflectors 1450 can further be configured to allow deflectors 1450 to collapse and expand with expandable body 1412 (e.g., to fit within a delivery catheter). The locations of deflectors 1450 can further be configured to prevent deflectors 1450 from occluding a vessel of chamber of heart H in which device 1400 is implanted. The locations of deflectors 1450 can further be configured to modify a hemodynamic characteristic (e.g., helicity, vorticity, velocity, turbulence, flow direction, etc.) of blood flowing into, through, or out of device 1400 in a particular manner.

In general, deflectors 1450 can take a number of different forms (i.e., shapes, sizes, etc.). Physical dimensions (e.g., length, width, shape, cross-sectional shape, etc.) of deflectors 1450 can be configured to prevent flow conditioner 1410A from interfering with other parts of device 1400 or adjacent tissue walls (e.g., tissue wall TW). In other examples, components of device 1400 can be designed to fit around deflectors 1450 or to permit deflectors 1450 to pass through. The physical dimension of deflectors 1450 can further be configured to allow deflectors 1450 to collapse and expand with expandable body 1412 (e.g., to fit within a delivery catheter). The physical dimensions of deflectors 1450 can further be configured to prevent deflectors 1450 from occluding a vessel of chamber of heart H in which device 1400 is implanted. That is, a length and/or width of deflectors 1450 can be relatively short enough so that deflectors 1450 do not protrude from device 1400 and extend fully across a vessel or chamber of heart H and block blood flow. The physical dimensions of deflectors 1450 can further be configured to modify a hemodynamic characteristic (e.g., helicity, vorticity, velocity, turbulence, flow direction, etc.) of blood flowing into, through, or out of device 1400 in a particular manner.

FIG. 20B shows shunt device 1400 including multi-direction deflector-type flow conditioner 1410B, body 1412, inflow end 1418, and outflow end 1420. Body 1412 is formed of struts 1422 and openings 1424. Body 1412 includes central flow tube 1426, flow path 1428, and arms 1430, and defines flow axis FL. Central flow tube 1426 includes inner surface 1432 and outer surface 1434. Flow conditioner 1410B is arranged in first set 1451C and second set 1451D and includes first direction deflector 1455A, second direction deflector 1455B, first direction deflector 1455C, and second direction deflector 1455D. First direction deflector 1455A includes first portion 1456A and second portion 1458A. Second direction deflector 1455B includes first portion 1456B and second portion 1458B. First direction deflector 1455C includes first portion 1456C and second portion 1458C. Second direction deflector 1455D includes first portion 1456D and second portion 1458D. FIG. 20B also shows tissue wall TW, arrow D1 indicating a first direction with respect to tissue wall TW, and arrow D2 indicating a second direction with respect to tissue wall TW.

In the example shown in FIG. 20B, flow conditioner 1410B includes first direction deflector 1455A, including first portion 1456A and second portion 1458A; second direction deflector 1455B, including first portion 1456B and second portion 1458B; first direction deflector 1455C, including first portion 1456C and second portion 1458C; and second direction deflector 1455D, including first portion 1456D and second portion 1458D. First set 1451C includes first direction deflector 1455A and second direction deflector 1455B connected to body 1412 adjacent inflow end 1418. Second set 1451D includes first direction deflector 1455C and second direction deflector 1455D connected to body 1412 adjacent outflow end 1420. Although device 1400 is shown in FIG. 20B as including first set 1451C adjacent inflow end 1418 and second set 1451D adjacent outflow end 1420, other examples can include either first set 1451C or second set 1451D instead of both sets.

At inflow end 1418, first direction deflector 1455A includes first portion 1456A connected to body 1412 and second portion 1458A connected to first portion 1456A. Using tissue wall TW as a reference plane, each of first portion 1456A and second portion 1458A are angled in the first direction with respect to tissue wall TW that is denoted by arrow D1. Second portion 1458A has a greater angle with respect to flow axis FL than first portion 1456A. Second direction deflector 1455B similarly includes first portion 1456B connected to body 1412 and second portion 1458B connected to first portion 1456B. Using tissue wall TW as a reference plane, each of first portion 1456B and second portion 1458B are angled in the second direction with respect to tissue wall TW that is denoted by arrow D2. Second portion 1458B has a greater angle with respect to flow axis FL than first portion 1456B.

At outflow end 1420, first direction deflector 1455C includes first portion 1456C connected to body 1412 and second portion 1458C connected to first portion 1456C. Using tissue wall TW as a reference plane, each of first portion 1456C and second portion 1458C are angled in the first direction with respect to tissue wall TW that is denoted by arrow D1. Second portion 1458C has a greater angle with respect to flow axis FL than first portion 1456C. Second direction deflector 1455D similarly includes first portion 1456D connected to body 1412 and second portion 1458D connected to first portion 1456D. Using tissue wall TW as a reference plane, each of first portion 1456D and second portion 1458D are angled in the second direction with respect to tissue wall TW that is denoted by arrow D2. Second portion 1458D has a greater angle with respect to flow axis FL than first portion 1456D.

FIG. 20C shows shunt device 1400 including multi-direction deflector-type flow conditioner 1410C, body 1412, inflow end 1418, and outflow end 1420. Body 1412 is formed of struts 1422 and openings 1424. Body 1412 includes central flow tube 1426, flow path 1428, and arms 1430, and defines flow axis FL. Central flow tube 1426 includes inner surface 1432 and outer surface 1434. Flow conditioner 1410C is arranged in first set 1451E and second set 1451F and includes curved first direction deflector 1460A, curved second direction deflector 1460B, curved first direction deflector 1460C, and curved second direction deflector 1460D. FIG. 20C also shows tissue wall TW, arrow D1 indicating a first direction with respect to tissue wall TW, and arrow D2 indicating a second direction with respect to tissue wall TW.

In the example shown in FIG. 20C, flow conditioner 1410C includes curved first direction deflector 1460A, curved second direction deflector 1460B, curved first direction deflector 1460C, and curved second direction deflector 1460D. First set 1451E includes curved first direction deflector 1460A and curved second direction deflector 1460B connected to body 1412 adjacent inflow end 1418. Second set 1451F includes curved first direction deflector 1460C and curved second direction deflector 1460D connected to body 1412 adjacent outflow end 1420. Curved first direction deflectors 1460A and 1460C are similar to first direction deflectors 1455A and 1455C shown in FIG. 20B, except curved first direction deflectors 1460A and 1460C are curved in the first direction with respect to tissue wall TW that is denoted by arrow D1. Curved second direction deflectors 1460B and 1460D are similar to second direction deflectors 1455B and 1455D shown in FIG. 20B, except curved second direction deflectors 1460B and 1460D are curved in the second direction with respect to tissue wall TW that is denoted by arrow D2. Each of curved first direction deflectors 1460A and 1460C and curved second direction deflectors 1460B and 1460D can have smooth or continuously curved profiles to direct blood flow into or out of device 1400.

Referring to all of FIGS. 20A-20C, once devices 1400 are implanted in the cardiovascular system (e.g., the inter-atrial septum or the tissue wall between the left atrium and the coronary sinus), circulating blood passes through devices 1400. As blood flows into, through, or out of devices 1400 along flow axis FL, flow conditioners 1410 (including first direction deflector 1450A and second direction deflector 1450B, first direction deflector 1455A and second direction deflector 1455B, and curved first direction deflector 1460A and curved second direction deflector 1460B) interact with the blood flow to modify or affect a hemodynamic characteristic (e.g., helicity, vorticity, velocity, turbulence, flow direction, etc.) of the flow. First sets 1451A, 1451C, and 1451E of flow conditioners 1410 interact with blood flowing into devices 1400 to direct blood flowing in multiple directions in a vessel or chamber of heart H into devices 1400 along flow axis FL. Second sets 1451B, 1451D, and 1451F of flow conditioners 1410 change the direction of the blood flow out of devices 1400 to direct the blood flow in multiple directions. For example, flow conditioners 1410 can direct the blood flow out of devices 1400 to join with a natural blood flow direction and/or pattern in a vessel or chamber of heart H. In some examples, flow conditioners 1410 can direct the flood flow along a tissue wall (e.g., tissue wall TW). In some examples, flow conditioners 1410 can align the flow with a natural vortical flow pattern of blood through a vessel or chamber of heart H, such as the left-sided flow vortex in left atrium LA or the right-sided flow vortex in right atrium RA. In other examples, flow conditioners 1410 can align the flow with a natural helical flow pattern of blood through a vessel or chamber of heart H, such as helical flow in coronary sinus CS.

In addition to the benefits described above with respect to devices 1100 and 1200, incorporating multi-direction deflector-type flow conditioners 1410 on devices 1400 allows for more precise tuning of flow alignment for blood flowing into and/or out of devices 1400. This can minimize disruption caused by the artificial flow path through shunt devices 1400 branching from a first natural flow path or pattern in a vessel or chamber of heart H and joining with a second natural flow path or pattern in a different vessel or chamber of heart H. Multi-direction deflector-type flow conditioners 1410 including first sets 1451A, 1451C, and 1451E at inflow end 1418 can mitigate venturi effects that may otherwise cause disruptions where blood flows into devices 1400. Moreover, the natural flow path in a chamber of heart H may include central vortices or helices, and multi-direction deflector-type flow conditioners 1410 including second sets 1451B, 1451D, and 1451F at outflow end 1420 can cause the blood flow out of devices 1400 to spread out and avoid jetting through the central flow patterns in the chamber, such as the right-sided flow vortex in right atrium RA, thereby minimizing disruption.

The multi-direction deflector-type flow conditioners described herein can take a number of different forms. Three examples are provided with reference to FIGS. 20A-20C. These examples are not intended to be limiting, and other examples are possible. Yet other examples can include combinations of the features shown in FIGS. 20A-20C or combinations of the features shown in FIGS. 19A-19C.

Although depicted in FIGS. 9-20C as separate examples, a cardiovascular shunt device according to techniques of this disclosure can include any combination of the foregoing features, unless expressly limited. Any of shunt devices 1100, 1200, 1300, and 1400 can maintain kinetic energy of the cardiovascular blood flow, which in turn reduces the cardiac work needed and improves cardiac efficiency.

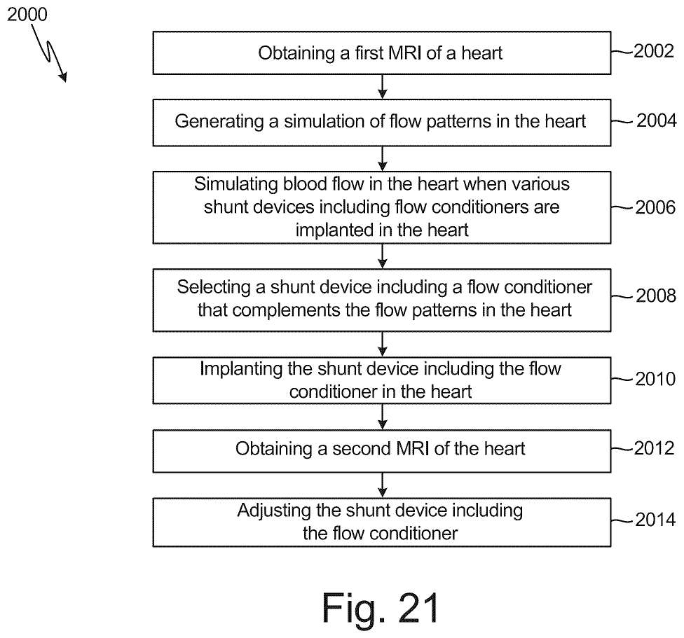

FIG. 21 is a flowchart showing method 2000 for selecting a shunt device including a flow conditioner for implantation in the heart. Method 2000 includes steps 2002-2014. A shunt device including a flow conditioner is selected according to method 2000 to minimize or eliminate disruption of or enhance flow patterns of blood flow in a heart of a patient.

Step 2002 includes obtaining a first MRI of a heart. The first MRI (magnetic resonance imaging) can visualize the flow patterns of blood flow in the heart of the patient. Specifically, the first MRI can visualize the flow patterns of blood flow in the right atrium, the left atrium, and/or the coronary sinus of the heart of the patient.

The first MRI can be a 4D MRI that visualizes the flow patterns of blood flow in the heart of a patient. Additionally, the 4D MRI can measure volumes in the chambers of the heart, sizes of the chambers of the heart, geometries of the chambers of the heart, compliances of the chambers of the heart, and/or blood pressures in the chambers of the heart. The 4D MRI can also track movement of the chambers of the heart and movement of the tricuspid valve (also known as tricuspid annular plan systolic excursion (TAPSE)).

Step 2004 includes generating a simulation of the flow patterns in the heart. The simulation of the flow patterns in the heart can be generated based on the first MRI. This allows the simulation to be patient specific. The simulation will simulate the flow patterns in the heart of the patient, and specifically in the right atrium, the left atrium, and the coronary sinus of the heart. The simulation can also simulate the volumes, sizes, geometries, compliances, and blood pressures of the chambers of the heart based on data from the first MRI. The simulation can be generated using any suitable software program.

Step 2006 includes simulating blood flow in the heart when various shunt devices including flow conditioners are implanted in the heart. The simulated blood flow in the heart is modulated by the shunt devices including the flow conditioners to simulate the impact of the shunt devices and the flow conditioners on the flow patterns in the heart. The blood flow in the heart can be simulated when the heart includes shunt devices including various types of flow conditioners (e.g., fin type, plate type, and/or deflector type) having varying forms (e.g., varying shapes, sizes, presence of flow microfeatures, etc.), varying physical dimensions, varying arrangements or positions with respect to the shunt device, varying attachment or extension angles, and/or other possible variations described herein.

Step 2008 includes selecting the shunt device including a flow conditioner (or multiple flow conditioners) that complements the flow patterns in the heart. The shunt device including the flow conditioner is selected to minimize or eliminate disruption of or enhance flow patterns in the heart. Specifically, step 2008 can include selecting a design of the shunt device including the flow conditioner that complements the flow patterns in the heart. More specifically, a type of the flow conditioner can be selected to complement the flow patterns in the heart; a form and/or physical dimension of the flow conditioner can be selected to complement the flow pattern in the heart; and an arrangement or position and/or angle of the flow conditioner can be selected to complement the flow pattern in the heart.

The right atrium of the heart has a right-sided flow vortex as a natural flow pattern in the heart. The design of the shunt device including the flow conditioner can be selected to complement the right-sided flow vortex in the right atrium of the heart. The left atrium of the heart has a left-sided flow vortex as a natural flow pattern in the heart. The design of the shunt device including the flow conditioner can be selected to complement the left-sided flow vortex in the left atrium of the heart. The coronary sinus has a helical flow pattern. The design of the shunt device including the flow conditioner can be selected to complement the helical flow pattern in the coronary sinus.

In an alternate example, step 2008 can include selecting a design of a shunt device including a flow conditioner that enhances the flow pattern in the right atrium of the heart and/or reestablishes the natural flow pattern in the right atrium of the heart. Specifically, if the patient has lost the right-sided flow vortex of blood flow in the right atrium of the heart due to age, disease, or anatomical defects, the design of the shunt device including the flow conditioner can be selected to reestablish the right-sided flow vortex of blood flow in the right atrium of the heart.

Step 2010 includes implanting the shunt device including the flow conditioner in the heart. The shunt device including the flow conditioner can be implanted using any suitable method. For example, the shunt device including the flow conditioner can be implanted according to methods described in U.S. Pat. No. 9,789,294, filed on Oct. 6, 2016, issued on Oct. 17, 2017, and entitled “Expandable Cardiac Shunt,” the disclosure of which is incorporated by reference in its entirety.

Step 2012 includes obtaining a second MRI of the heart. The second MRI (magnetic resonance imaging) can visualize the flow patterns of blood flow in the heart of the patient after the shunt device including the flow conditioner has been implanted. Specifically, the second MRI can visualize the flow patterns of blood flow in the right atrium, the left atrium, and/or the coronary sinus of the heart of the patient after the shunt device including the flow conditioner has been implanted.

The second MRI can be a 4D MRI that visualizes the flow patterns of blood flow in the heart of a patient after the shunt device including the flow conditioner has been implanted. Additionally, the 4D MRI can measure volumes in the chambers of the heart, sizes of the chambers of the heart, geometries of the chambers of the heart, compliances of the chambers of the heart, and/or blood pressures in the chambers of the heart. The 4D MRI can also track movement of the chambers of the heart and movement of the tricuspid valve (also known as tricuspid annular plan systolic excursion (TAPSE)).

The second MRI is obtained to confirm that the shunt device including the flow conditioner complements (e.g., has minimal to no disruption of or enhances) the flow patterns in the heart. Further, the second MRI can be obtained to determine whether the shunt device including the flow conditioner has enhanced and/or reestablished the natural flow patterns in the heart. Specifically, the second MRI can be obtained to determine whether the shunt device including the flow conditioner has reestablished a right-sided flow vortex in a right atrium of the heart.

The second MRI can also confirm the overall health of the heart after the shunt device including the flow conditioner has been implanted. Specifically, the volumes in the chambers of the heart, sizes of the chambers of the heart, geometries of the chambers of the heart, compliances of the chambers of the heart, and/or blood pressures in the chambers of the heart from the second MRI can be compared to the same readings from the first MRI to confirm overall health of the heart. In one example, the volumes, sizes, geometries, and/or compliances of the chambers of the heart can be analyzed to determine if the left side of the heart has experienced remodeling (shrinkage) due to the reduced blood pressure on the left side of the heart after the shunt device including the flow conditioner has been implanted. Further, the volumes, sizes, geometries, and/or compliances of the chambers of the heart can be analyzed to determine if the right side of the heart is being overloaded due to the increased blood pressure in the right side of the heart.

Step 2014 includes adjusting the shunt device including the flow conditioner. The shunt device including the flow conditioner can be adjusted if the second MRI shows that the implantation of the shunt device including the flow conditioner has not had the desired effect on the flow patterns in or overall health of the heart. In one example, a type of the flow conditioner can be changed, such as from one or more fin type, plate type, or deflector type flow conditioners or a combination of types to one or more of a different type of flow conditioner or a different combination of types. In another example, a form, physical dimension, and/or arrangement or position of the flow conditioner can be adjusted. In another example, the angle of the flow conditioner can be adjusted. For example, the flow conditioner can be connected to the shunt device by an adjustable bias member that can be adjusted such that the flow conditioner has a different angle, or the flow conditioner can be electromechanically actuated to a different angle.

Method 2000 as described herewith can be used to aid in the selection and implantation of any suitable shunt device including a flow conditioner. In one example, method 2000 can be used to aid in the selection and implantation of shunt device 100 (shown in FIGS. 6A-8), shunt device 1100 (shown in FIGS. 9-15), shunt device 1200 (shown in FIGS. 16-18E), shunt device 1300 (shown in FIGS. 19A-19C), or shunt device 1400 (shown in FIGS. 20A-20C). Method 2000 can be used to select a design of shunt device 100, 1100, 1200, 1300, or 1400 that will complement (e.g., minimize or eliminate disruptions or enhance) the flow patterns in the heart. In alternate examples, method 2000 can be used with any other design of a shunt device including a flow conditioner.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.). That is, any of shunt devices 100, 1100, 1200, 1300, and 1400 or components of shunt devices 100, 1100, 1200, 1300, and 1400 can be sterilized before being delivered into the body.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Discussion of Possible Examples

The following are non-exclusive descriptions of possible examples of the present invention.

A shunt device includes a shunt body formed of a plurality of struts and a flow conditioner connected to the plurality of struts of the shunt body. The shunt body includes a central flow tube, a flow path extending through the central flow tube, and a plurality of arms extending outward from the central flow tube and configured to secure the shunt device to a tissue wall. The flow conditioner is positioned to modify a hemodynamic characteristic of a flow of blood through or out of the central flow tube.

The shunt device of the preceding paragraph can optionally include, additionally and/or alternatively, any one or more of the following features, configurations, and/or additional components:

The flow conditioner can include one or more fins.

At least one of the one or more fins can be connected to one of the plurality of arms.

The one or more fins can be connected circumferentially about the central flow tube.

The one or more fins can be connected to ones of the plurality of struts that form a first row of openings in the central flow tube, the first row of openings being adjacent to an inflow end of the central flow tube.

The one or more fins can be connected to ones of the plurality of struts that form a second row of openings in the central flow tube, the second row of openings being adjacent to an outflow end of the central flow tube.

The one or more fins can be angled radially inward from a circumference of the central flow tube.

The one or more fins can be connected adjacent to an inflow or outflow end of the central flow tube.

The one or more fins can be angled radially inward from the inflow or outflow end of the central flow tube.

The one or more fins can include a first fin connected to the shunt body at an inflow end of the shunt device to modify the hemodynamic characteristic of the flow of blood through the central flow tube and a second fin connected to the shunt body at an outflow end of the shunt device to modify the hemodynamic characteristic of the flow of blood out of the central flow tube.

The one or more fins can be deflectable by the flow of blood through or out of the central flow tube.

The one or more fins can be connected to the shunt body by a spring.

The one or more fins can be airfoils, and the one or more fins can include flow microfeatures proximal to a leading edge of respective ones of the one or more fins.

The flow microfeatures can include at least one of a vortex generator, a leading edge notch, a leading edge dogtooth, a boundary layer fence, and a vortilon.

The flow conditioner and the shunt body can form a monolithic structure.

The flow conditioner and a portion of the shunt body to which the flow conditioner is connected can be formed of a shape-memory alloy.

The shape-memory alloy can be nitinol.

The flow conditioner can be electromechanically actuated.

An attachment angle of the flow conditioner as measured with respect to a longitudinal axis of a respective one of the plurality of struts to which the flow conditioner is connected can be controllable over a range of possible angles.

The flow conditioner can include one or more deflectors that are connected adjacent to an outflow end of the central flow tube and positioned to direct the flow of blood out of the central flow tube in a first direction.

The one or more deflectors can extend parallel to a flow axis through the central flow tube.

The one or more deflectors can include a primary deflector that further includes a first portion that extends parallel to a flow axis through the central flow tube and a second portion that is angled toward the flow axis in the first direction.

The one or more deflectors can include a primary deflector that is curved toward a

flow axis through the central flow tube in the first direction.

The flow conditioner can include deflectors that are connected at an outflow end of the device and positioned to direct the flow of blood out of the central flow tube in a first direction and a second direction.

The deflectors can extend across the central flow tube.

The deflectors can be angled radially outward with respect to a flow axis through the central flow tube.

The deflectors can include a first deflector that is angled in the first direction and a second deflector that is angled in the second direction.

The deflectors can be curved.

The deflectors can include a first deflector that is curved in the first direction and a second deflector that is curved in the second direction.

The flow conditioner can include one or more deflectors that are connected adjacent to an inflow end of the central flow tube and positioned to direct the flow of blood into the central flow tube along a flow axis through the central flow tube.

The flow conditioner can include one or more plates that span a portion of the central flow tube such that the one or more plates intersect a flow axis through the central flow tube, the one or more plates each including a plurality of flow passages.

The one or more plates can include a first plate having a first plurality of flow passages and a second plate having a second plurality of flow passages, and the first plate can be connected at an inflow end of the shunt device to modify the hemodynamic characteristic of the flow of blood through the central flow tube and the second plate can be connected to an outflow end of the shunt device to modify the hemodynamic characteristic of the flow of blood out of the central flow tube.

The flow conditioner can be connected to the shunt body at an inflow end of the shunt device to modify the hemodynamic characteristic of the flow of blood through the central flow tube.

The flow conditioner can be connected to the shunt body at an outflow end of the shunt device to modify the hemodynamic characteristic of the flow of blood out of the central flow tube.

The flow conditioner can include a first flow conditioner feature connected to the shunt body at an inflow end of the shunt device and a second flow conditioner feature connected to the shunt body at an outflow end of the shunt device, and the first flow conditioner feature is positioned to modify the hemodynamic characteristic of the flow of blood through the central flow tube and the second flow conditioner feature is positioned to modify the hemodynamic characteristic of the flow of blood out of the central flow tube.

The flow conditioner can have a physical dimension that causes the flow conditioner to avoid interaction with the tissue wall and/or occlusion of a blood vessel.

The flow conditioner can include at least one of a fin, a deflector, and a plate that includes a plurality of flow passages.

The shunt device can be sterilized.

The shunt device can be positioned in a tissue wall between a left atrium and a coronary sinus of a heart.

The shunt device can be positioned in a tissue wall between a left atrium and a right atrium of a heart.

While the invention has been described with reference to an exemplary example(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular example(s) disclosed, but that the invention will include all examples falling within the scope of the appended claims.

1. A shunt device comprising:
a shunt body formed of a plurality of struts, the shunt body comprising:
a central flow tube;
a flow path extending through the central flow tube; and
a plurality of arms extending outward from the central flow tube and configured to secure the shunt device to a tissue wall; and
a flow conditioner connected to the plurality of struts of the shunt body, the flow conditioner being positioned to modify a hemodynamic characteristic of a flow of blood through or out of the central flow tube.

2. The shunt device of claim 1, wherein the flow conditioner includes one or more fins.

3. The shunt device of claim 2, wherein at least one of the one or more fins is connected to one of the plurality of arms.

4. The shunt device of claim 2, wherein the one or more fins are connected circumferentially about the central flow tube.

5. The shunt device of claim 4, wherein the one or more fins are connected to ones of the plurality of struts that form a first row of openings in the central flow tube, the first row of openings being adjacent to an inflow end of the central flow tube.

6. The shunt device of claim 4, wherein the one or more fins are connected to ones of the plurality of struts that form a second row of openings in the central flow tube, the second row of openings being adjacent to an outflow end of the central flow tube.

7. The shunt device of claim 4, wherein the one or more fins are angled radially inward from a circumference of the central flow tube.

8. The shunt device of claim 2, wherein the one or more fins are deflectable by the flow of blood through or out of the central flow tube.

9. The shunt device of claim 2, wherein the one or more fins are airfoils, and wherein the one or more fins include flow microfeatures proximal to a leading edge of respective ones of the one or more fins.

10. The shunt device of claim 1, wherein the flow conditioner includes one or more plates that span a portion of the central flow tube such that the one or more plates intersect a flow axis through the central flow tube, the one or more plates each including a plurality of flow passages.

11. The shunt device of claim 1, wherein the flow conditioner is connected to the shunt body at an inflow end of the shunt device to modify the hemodynamic characteristic of the flow of blood through the central flow tube and/or wherein the flow conditioner is connected to the shunt body at an outflow end of the shunt device to modify the hemodynamic characteristic of the flow of blood out of the central flow tube.

12. The shunt device of claim 1, wherein the flow conditioner includes at least one of a fin, a deflector, and a plate that includes a plurality of flow passages.

13. The shunt implant device of claim 1, wherein the shunt device is sterilized.

14. The shunt implant device of claim 1, wherein the shunt device is positioned in a tissue wall between a left atrium and a coronary sinus of a heart or in a tissue wall between the left atrium and a right atrium of the heart.

15. A shunt device comprising:
a shunt body formed of a plurality of struts, the shunt body comprising:
a central flow tube;
a flow path extending through the central flow tube; and
a plurality of arms extending outward from the central flow tube and configured to secure the shunt device to a tissue wall; and
a flow conditioner connected to the plurality of struts of the shunt body, the flow conditioner being positioned to modify a hemodynamic characteristic of a flow of blood through or out of the central flow tube;
wherein the flow conditioner and the shunt body form a monolithic structure.

16. The shunt device of claim 15, wherein the flow conditioner and a portion of the shunt body to which the flow conditioner is connected are formed of a shape-memory alloy.

17. The shunt device of claim 16, wherein the shape-memory alloy is nitinol.

18. A shunt device comprising:
a shunt body formed of a plurality of struts, the shunt body comprising:
a central flow tube;
a flow path extending through the central flow tube; and
a plurality of arms extending outward from the central flow tube and configured to secure the shunt device to a tissue wall; and
a flow conditioner connected to the plurality of struts of the shunt body, the flow conditioner being positioned to modify a hemodynamic characteristic of a flow of blood through or out of the central flow tube;
wherein the flow conditioner is electromechanically actuated.

19. The shunt device of claim 18, wherein a bias member extends between the flow conditioner and one or more struts of the plurality of struts.

20. The shunt device of claim 19, wherein the bias member is a spring.
